(19) United States
(12) Patent Application Publication
(10) Pub. No.: US 2023/0117995 A1
(43) Pub. Date: Apr. 20, 2023

(54) CONSUMABLE ELECTROLYTE COMPOSITIONS AND METHODS OF USE THEREOF

(71) Applicant: Krampade, LLC, Lincoln, NE (US)

(72) Inventors: Cameron MURPHY, Lincoln, NE (US); Eric J. MURPHY, Lincoln, NE (US); Sean MURPHY, Lincoln, NE (US)

(73) Assignee: Krampade, LLC, Lincoln, NE (US)

(21) Appl. No.: 17/874,949

(22) Filed: Jul. 27, 2022

Related U.S. Application Data
(63) Continuation of application No. 15/625,993, filed on Jun. 16, 2017, now abandoned.
(60) Provisional application No. 62/351,855, filed on Jun. 17, 2016.

(51) Int. Cl. A23L 33/16; A23L 2/68; A23L 2/60; A23L 2/38; A23L 2/52

(52) U.S. Cl. CPC A23L 33/16; A23L 2/68; A23L 2/60; A23L 2/38; A23L 2/52; A23V 2002/00

(57) ABSTRACT

The present disclosure relates generally to consumable electrolyte compositions, and more specifically to such compositions that contain high concentrations of potassium for alleviation of muscle cramping.

### CROSS REFERENCE TO RELATED APPLICATION

This application claims the benefit of U.S. Provisional Application Ser. No. 62/351,855, filed Jun. 17, 2016, which is incorporated herein by reference in its entirety.

### FIELD

The present disclosure relates generally to consumable electrolyte compositions, and more specifically to such compositions that contain high concentrations of potassium for alleviation of muscle cramping.

### BACKGROUND

Sports beverages are becoming increasingly popular among the general public as consumer preference continues to shift away from sugar-rich beverages, such as carbonated sodas, to healthier alternatives for fluid beverages. Sports drinks in particular are typically used as electrolyte-replacement and rehydration beverages. Electrolytes are charged molecules, such as for example sodium (Na⁺), potassium (K⁺), calcium (Ca²⁺), magnesium (Mg²⁺), chloride (Cl⁻), hydrogen phosphate (HPO₄²⁻), and hydrogen carbonate (HCO₃⁻), that allow an aqueous solution to conduct electricity.

Electrolytes are commonly found in bodily fluids and play pivotal roles in nerve and muscle function, among other bodily functions. Electrolytes may travel through cell membranes to either enter or exit a cell via ion channels. For example, muscle contraction involves the presence of calcium, sodium, and potassium. Maintaining electrolyte balance in the body is important for maintaining normal bodily functions. Electrolyte balance is controlled by various hormones, and electrolytes are excreted via urine following processing in the kidney, as well as via sweat. If sufficient levels of electrolytes are not available, muscle weakness or muscle cramping may occur, in addition to other undesirable symptoms.

Athletes and the elderly are examples of individuals at risk of experiencing electrolyte imbalance and muscle cramping. Profuse sweating during athletic activity leads to loss of key electrolytes through the sweat, predominantly sodium and to a lesser extent potassium. Under these conditions, low blood sodium levels results in increased aldosterone release from the adrenal gland, shifting the kidney from wasting sodium to wasting of potassium. This exacerbates the loss of potassium from muscle and other depolarizable tissues. If these electrolytes are not sufficiently replaced, athletic performance will decline as muscle function decreases and cramping ensues. Muscle cramping in particular is predominantly due to a reduction of potassium and under extreme circumstances a reduction in calcium available to muscles. In the elderly, declining muscle function associated with increasing age and electrolyte imbalance, as well as increased susceptibility to dehydration, can lead to muscle cramping.

Electrolyte-replacement beverages, such as sports drinks, are thus used to hydrate the body and replenish the supply of key electrolytes. This is of particular importance to athletes who wish to maintain optimal athletic performance. Commercially available electrolyte replacement beverages are typically formulated to mimic the contents of sweat or blood plasma, and predominantly contain sodium and glucose or other suitable sugar.

While these commercial beverages often also contain potassium, which is another component of sweat and whose depletion from the body can lead to muscle cramping, the concentration of potassium in these beverages is generally relatively low. The leading products in the sports drink industry (e.g. Gatorade, Powerade, and Body Armor), as well as potassium-replacement products (e.g. coconut water), contain an insufficient amount of potassium to reliably act as either a prophylactic against muscle cramping, or to provide a rapid means to mitigate acute muscle cramps.

Supplementing electrolyte-replacement beverages with high levels of potassium has been discouraged for a variety of reasons. First, high levels of potassium in beverages are associated with a bitter or metallic taste, which is undesirable. The addition of sugars has been suggested to mask the poor taste, but this sugar addition results in increasing calorie content of the beverage, which is also undesirable. Further, potassium has been regarded as a nutrient that may promote gastric mobility, which would be undesirable during an athletic performance or in other situations. Finally, as the kidney is responsible in part for maintaining potassium balance in the body, poor kidney function can lead to decreased ability to flush excess potassium from the body which may lead to a dangerous condition known as hyperkalemia. Thus, a high level of potassium in the body is a concern for those individuals with poor kidney function.

However, commercially available electrolyte-replacement beverages are consistently ineffective at alleviating muscle cramping, particularly severe or chronic muscle cramping. Thus, there exists a need for an electrolyte-replacement beverage that contains a sufficiently high concentration of potassium and that alleviates muscle cramping.

### BRIEF SUMMARY

In one aspect, the present disclosure relates to a consumable electrolyte composition including: a) potassium in the range of about 2 g/L to about 8 g/L; b) chloride in the range of about 2 g/L to about 8 g/L; and c) an organic acid in an amount sufficient to generate a pH in the range of about 3.0 to about 3.5, where the potassium is present in the range of about 45% to about 55% of the total dry weight of the potassium and the chloride in the composition. In some embodiments, the potassium is in the range of about 2 g/L to about 4 g/L. In some embodiments, the potassium is in the range of about 4 g/L to about 6 g/L. In some embodiments, the potassium is in the range of about 6 g/L to about 8 g/L. In some embodiments that may be combined with any of the preceding embodiments, the chloride is in the range of about 2 g/L to about 4 g/L. In some embodiments that may be combined with any of the preceding embodiments, the chloride is in the range of about 4 g/L to about 6 g/L. In some embodiments that may be combined with any of the preceding embodiments, the chloride is in the range of about 6 g/L to about 8 g/L. In some embodiments that may be combined with any of the preceding embodiments, the organic acid is in the range of about 1 g/L to about 3 g/L. In some embodiments that may be combined with any of the preceding embodiments, the organic acid is citric acid. In some embodiments that may be combined with any of the preceding embodiments, the composition further includes one or more additional electrolytes. In some embodiments, the electrolyte is sodium. In some embodiments, the sodium is in the range of about 0.25 g/L to about 2 g/L. In some embodiments, the electrolyte is magnesium. In some embodiments, the source of magnesium is magnesium chloride. In some embodiments, the magnesium is in the range of about 0.15 g/L to about 0.25 g/L. In some embodiments, the magnesium is in the range of about 0.25 g/L to about 0.35 g/L. In some embodiments, the magnesium is in the range of about 0.35 g/L to about 0.45 g/L. In some embodiments that may be combined with any of the preceding embodiments, the composition further includes a sweetener. In some embodiments, the sweetener is sucrose.

In another aspect, the present disclosure relates to a method of alleviating muscle cramping in a subject, the method including administering to the subject a consumable electrolyte composition of any of the preceding embodiments in an amount sufficient to alleviate the muscle cramp. In some embodiments, the amount is in the range of about 0.3 L to about 0.6 L. In some embodiments, the amount is about 0.5 L. In some embodiments that may be combined with any of the preceding embodiments, the composition is administered over a time interval. In some embodiments that may be combined with any of the preceding embodiments, the composition is administered about once daily. In some embodiments that may be combined with any of the preceding embodiments, the muscle cramp is a leg cramp, a foot cramp, or a menstrual cramp. In some embodiments that may be combined with any of the preceding embodiments, alleviation includes reducing the duration of the muscle cramp and/or reducing the severity of the muscle cramp. In some embodiments that may be combined with any of the preceding embodiments, the subject is a human. In some embodiments, the human subject is an athlete or an elderly individual. In some embodiments, the human subject is a female.

In yet another aspect, the present disclosure relates to a method of alleviating menstrual cramping in a subject, the method including administering to the subject a consumable electrolyte composition of any of the preceding embodiments in an amount sufficient to alleviate the menstrual cramp. In some embodiments, the amount is in the range of about 0.3 L to about 0.6 L. In some embodiments, the amount is about 0.5 L. In some embodiments that may be combined with any of the preceding embodiments, the composition is administered over a time interval. In some embodiments that may be combined with any of the preceding embodiments, the composition is administered about once daily. In some embodiments that may be combined with any of the preceding embodiments, alleviation includes reducing the duration of the menstrual cramp and/or reducing the severity of the menstrual cramp. In some embodiments that may be combined with any of the preceding embodiments, the subject is a human. In some embodiments, the human subject is a female individual. In some embodiments that may be combined with any of the preceding embodiments, alleviation of cramping in a subject occurs from about 30 seconds to about 5 minutes after administering the consumable electrolyte composition.

### BRIEF DESCRIPTION OF THE DRAWINGS

The patent or application file contains at least one drawing executed in color. Copies of this patent or patent application publication with color drawings will be provided by the Office upon request and payment of the necessary fee.

The effectiveness of the consumable electrolyte compositions at reducing severity of cramping could be better understood with the illustration of the following figures. The size of circles in FIG. 2 to FIG. 6 represents the number of respondent individuals at a given level of cramping severity. In FIG. 1 to FIG. 6, blue indicates severity of cramping before the subjects became consumers of the consumable electrolyte compositions, and red indicates severity of cramping (if cramps did occur) after the subjects became consumers of the consumable electrolyte compositions.

FIG. 1 illustrates the surveyed average cramping severity by type of cramps. X-axis is the type of cramps. Y-axis is the cramping severity on a scale of 0-10, with 0 indicating no cramping and 10 indicating debilitating cramping.

FIG. 2 illustrates the general cramping severity ratings before and after the surveyed subjects became consumers of the consumable electrolyte compositions.

FIG. 3 illustrates the repetitive use cramping severity ratings before and after the surveyed subjects became consumers of the consumable electrolyte compositions.

FIG. 4 illustrates the nighttime leg and foot cramping severity ratings before and after the surveyed subjects became consumers of the consumable electrolyte compositions.

FIG. 5 illustrates exercise-induced cramping severity ratings before and after the surveyed subjects became consumers of the consumable electrolyte compositions.

FIG. 6 illustrates menstrual cramping severity ratings before and after the surveyed subjects became consumers of the consumable electrolyte compositions.

FIG. 7 illustrates the various additional positive attributes of the consumable electrolyte compositions according to surveyed respondents. X-axis is the type of positive attributes of the consumable electrolyte composition. Y-axis is the number of respondents in a given attribute category.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

The present disclosure relates generally to consumable electrolyte compositions, and more specifically to such compositions that contain high concentrations of potassium for alleviation of muscle cramping.

The present disclosure is based, at least in part, on Applicant's development of consumable electrolyte compositions containing a higher concentration of potassium than other commercially-available products. Applicant's compositions concomitantly contain a high concentration of chloride and have a relatively low pH. Surprisingly, Applicant has found that these electrolyte compositions do not have the bitter or metallic taste typically associated with high-potassium compositions. Further, Applicant has surprisingly found that these electrolyte compositions are very effective at alleviating muscle cramps in individuals who have consumed these compositions. Applicant's compositions act to prevent the onset of muscle cramping and provide rapid relief to individuals experiencing an acute muscle cramping episode.

Accordingly, disclosed herein are consumable electrolyte compositions for use in alleviating muscle cramps, as well as methods of administering these compositions to a subject. The compositions of the present disclosure contain potassium, chloride, and an organic acid, and may be used in methods of alleviating muscle cramps in a subject.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

Reference to “about” a value or parameter herein refers to the usual error range for the respective value readily known to the skilled person in this technical field. Reference to “about” a value or parameter herein includes (and describes) aspects that are directed to that value or parameter per se. For example, description referring to “about X” includes description of “X.”

It is understood that aspects and embodiments of the present disclosure described herein include “comprising,” “consisting,” and “consisting essentially of” aspects and embodiments.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### Consumable Electrolyte Compositions

The present disclosure provides consumable electrolyte compositions for alleviating muscle cramps. In some aspects, compositions of the present disclosure contain potassium, chloride, and an organic acid. Compositions as described herein may be administered to a subject to alleviate muscle cramping.

Potassium

In one aspect, consumable electrolyte compositions as described herein contain potassium. Potassium is a chemical element, abbreviated by the symbol “K” and having atomic number 19 on the period table of elements. In aqueous solution, potassium is a cation with a +1 charge (K⁺). Potassium is an important electrolyte that is present in bodily fluids. Various potassium salts may be used as a source of potassium to formulate compositions as described herein. In solution, these salts disassociate to form the potassium ion and the corresponding anion present in the salt. Suitable potassium salts for use in formulating compositions described herein include, for example, potassium chloride (KCl) and potassium citrate. Various potassium salts are commercially available and suitable potassium salts for use in the compositions and methods described herein would be readily apparent to one of skill in the art in view of the present disclosure. Additional common forms of dissociable potassium are potassium phosphate salts.

The quantity of potassium in compositions of the present disclosure may include, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, or about 80% to about 85% of the total dry weight of the components in the composition.

Compositions of the present disclosure may contain a potassium concentration of, for example, at least about 0.5 g/L, at least about 0.75 g/L, at least about 1 g/L, at least about 1.5 g/L, at least about 2 g/L, at least about 2.25 g/L, at least about 2.5 g/L, at least about 2.75 g/L, at least about 3 g/L, at least about 3.25 g/L, at least about 3.5 g/L, at least about 3.75 g/L, at least about 4 g/L, at least about 4.25 g/L, at least about 4.5 g/L, at least about 4.75 g/L, at least about 5 g/L, at least about 5.25 g/L, at least about 5.5 g/L, at least about 5.75 g/L, at least about 6 g/L, at least about 6.25 g/L, at least about 6.5 g/L, at least about 6.75 g/L, at least about 7 g/L, at least about 7.25 g/L, at least about 7.5 g/L, at least about 7.75 g/L, at least about 8 g/L, at least about 8.25 g/L, at least about 8.5 g/L, at least about 8.75 g/L, at least about 9 g/L, at least about 9.25 g/L, at least about 9.5 g/L, at least about 9.75 g/L, at least about 10 g/L, at least about 10.25 g/L, at least about 10.5 g/L, at least about 10.75 g/L, at least about 11 g/L, at least about 11.25 g/L, at least about 11.5 g/L, at least about 11.75 g/L, at least about 12 g/L, at least about 12.5 g/L, at least about 13 g/L, at least about 13.5 g/L, at least about 14 g/L, at least about 14.5 g/L, or at least about 15 g/L.

Compositions of the present disclosure may contain a potassium concentration of, for example, about 0.5 g/L to about 2.5 g/L, about 1 g/L to about 3 g/L, about 1.5 g/L to about 3.5 g/L, about 2 g/L to about 4 g/L, about 2.5 g/L to about 4.5 g/L, about 3 g/L to about 5 g/L, about 3.5 g/L to about 5.5 g/L, about 4 g/L to about 6 g/L, about 4.5 g/L to about 6.5 g/L, about 5 g/L to about 7 g/L, about 5.5 g/L to about 7.5 g/L, about 6 g/L to about 8 g/L, about 6.5 g/L to about 8.5 g/L, about 7 g/L to about 9 g/L, about 7.5 g/L to about 9.5 g/L, about 8 g/L to about 10 g/L, about 10.5 g/L to about 12.5 g/L, about 11 g/L to about 13 g/L, or about 13 g/L to about 15 g/L.

Compositions of the present disclosure may contain a potassium concentration of, for example, about 0.5 g/L to about 1 g/L, about 0.5 g/L to about 2 g/L, about 0.5 g/L to about 3 g/L, about 0.5 g/L to about 4 g/L, about 0.5 g/L to about 5 g/L, about 0.5 g/L to about 6 g/L, about 0.5 g/L to about 7 g/L, about 0.5 g/L to about 8 g/L, about 0.5 g/L to about 9 g/L, about 0.5 g/L to about 10 g/L, about 1 g/L to about 2 g/L, about 1 g/L to about 3 g/L, about 1 g/L to about 4 g/L, about 1 g/L to about 5 g/L, about 1 g/L to about 6 g/L, about 1 g/L to about 7 g/L, about 1 g/L to about 8 g/L, about 1 g/L to about 9 g/L, about 1 g/L to about 10 g/L, about 1.5 g/L to about 2 g/L, about 1.5 g/L to about 3 g/L, about 1.5 g/L to about 4 g/L, about 1.5 g/L to about 5 g/L, about 1.5 g/L to about 6 g/L, about 1.5 g/L to about 7 g/L, about 1.5 g/L to about 8 g/L, about 1.5 g/L to about 9 g/L, about 1.5 g/L to about 10 g/L, about 2 g/L to about 3 g/L, about 2 g/L to about 4 g/L, about 2 g/L to about 5 g/L, about 2 g/L to about 6 g/L, about 2 g/L to about 7 g/L, about 2 g/L to about 8 g/L, about 2 g/L to about 9 g/L, about 2 g/L to about 10 g/L, about 2.5 g/L to about 4 g/L, about 2.5 g/L to about 5 g/L, about 2.5 g/L to about 6 g/L, about 2.5 g/L to about 7 g/L, about 2.5 g/L to about 8 g/L, about 2.5 g/L to about 9 g/L, or about 2.5 g/L to about 10 g/L.

Compositions of the present disclosure may contain a potassium concentration of, for example, about 3 g/L to about 4 g/L, about 3 g/L to about 5 g/L, about 3 g/L to about 6 g/L, about 3 g/L to about 7 g/L, about 3 g/L to about 8 g/L, about 3 g/L to about 9 g/L, about 3 g/L to about 10 g/L, about 3.5 g/L to about 4 g/L, about 3.5 g/L to about 5 g/L, about 3.5 g/L to about 6 g/L, about 3.5 g/L to about 7 g/L, about 3.5 g/L to about 8 g/L, about 3.5 g/L to about 9 g/L, about 3.5 g/L to about 10 g/L, about 4 g/L to about 5 g/L, about 4 g/L to about 6 g/L, about 4 g/L to about 7 g/L, about 4 g/L to about 8 g/L, about 4 g/L to about 9 g/L, about 4 g/L to about 10 g/L, about 4.5 g/L to about 5 g/L, about 4.5 g/L to about 6 g/L, about 4.5 g/L to about 7 g/L, about 4.5 g/L to about 8 g/L, about 4.5 g/L to about 9 g/L, about 4.5 g/L to about 10 g/L, about 5 g/L to about 6 g/L, about 5 g/L to about 7 g/L, about 5 g/L to about 8 g/L, about 5 g/L to about 9 g/L, about 5 g/L to about 10 g/L, about 5.5 g/L to about 6 g/L, about 5.5 g/L to about 7 g/L, about 5.5 g/L to about 8 g/L, about 5.5 g/L to about 9 g/L, about 5.5 g/L to about 10 g/L, about 6 g/L to about 7 g/L, about 6 g/L to about 8 g/L, about 6 g/L to about 9 g/L, or about 6 g/L to about 10 g/L.

Compositions of the present disclosure may contain a potassium concentration of, for example, about 6.5 g/L to about 7 g/L, about 6.5 g/L to about 8 g/L, about 6.5 g/L to about 9 g/L, about 6.5 g/L to about 10 g/L, about 6.5 g/L to about 11 g/L, about 6.5 g/L to about 12 g/L, about 6.5 g/L to about 13 g/L, about 6.5 g/L to about 14 g/L, about 6.5 g/L to about 15 g/L, about 7 g/L to about 8 g/L, about 7 g/L to about 9 g/L, about 7 g/L to about 10 g/L, about 7 g/L to about 11 g/L, about 7 g/L to about 12 g/L, about 7 g/L to about 13 g/L, about 7 g/L to about 14 g/L, about 7 g/L to about 15 g/L, about 7.5 g/L to about 8 g/L, about 7.5 g/L to about 9 g/L, about 7.5 g/L to about 10 g/L, about 7.5 g/L to about 11 g/L, about 7.5 g/L to about 12 g/L, about 7.5 g/L to about 13 g/L, about 7.5 g/L to about 14 g/L, about 7.5 g/L to about 15 g/L, about 8 g/L to about 9 g/L, about 8 g/L to about 10 g/L, about 8 g/L to about 11 g/L about 8 g/L to about 12 g/L, about 8 g/L to about 13 g/L, about 8 g/L to about 14 g/L about 8 g/L to about 15 g/L, about 8.5 g/L to about 9 g/L, about 8.5 g/L to about 10 g/L, about 8.5 g/L to about 11 g/L, about 8.5 g/L to about 12 g/L, about 8.5 g/L to about 13 g/L, about 8.5 g/L to about 14 g/L, about 8.5 g/L to about 15 g/L, about 9 g/L to about 10 g/L, about 9 g/L to about 11 g/L, about 9 g/L to about 12 g/L, about 9 g/L to about 13 g/L, about 9 g/L to about 14 g/L, about 9 g/L to about 15 g/L, about 9.5 g/L to about 10 g/L, about 9.5 g/L to about 11 g/L, about 9.5 g/L to about 12 g/L, about 9.5 g/L to about 13 g/L, about 9.5 g/L to about 14 g/L, or about 9.5 g/L to about 15 g/L.

As described further herein, compositions of the present disclosure also contain chloride. Compositions of the present disclosure may contain a potassium concentration of, for example, about 25% to about 35%, about 30% to about 35%, about 35% to about 40%, about 40% to about 45%, about 45% to about 50%, about 50% to about 55%, about 55% to about 60%, about 60% to about 65%, or about 65% to about 70% of the total dry weight of the potassium and the chloride in the composition.

Chloride

In one aspect, consumable electrolyte compositions as described herein contain chloride. Chloride (Cl⁻) is the anion of chlorine, which is a chemical element abbreviated by the symbol “Cl” and having atomic number 17 on the period table of elements. In aqueous solution, chloride is an anion with a −1 charge (Cl⁻). Chloride is an important electrolyte that is present in bodily fluids. Chloride is thought to play an important role in the hydration of subjects administered the compositions as described herein, as the chloride plays an important role in driving water entry into cells. Various chloride salts may be used as a source of chloride to formulate compositions as described herein. In solution, these salts disassociate to form the chloride ion and the corresponding cation present in the salt. Suitable chloride salts for use in formulating compositions described herein include, for example, potassium chloride (KCl) and sodium chloride (NaCl). Various chloride salts are commercially available and suitable chloride salts for use in the compositions and methods described herein would be readily apparent to one of skill in the art in view of the present disclosure. Additional chloride salts, such as magnesium chloride, are not compatible for enhancing chloride in a meaningful way due to their cathartic effect on gastric mobility. However, under certain circumstances, the use of magnesium chloride (MgCl₂) may be desired. For instance, magnesium chloride may be used in a formulation to mask the saltiness of potassium chloride that may be displeasing to human individuals. For another instance, magnesium chloride may be added to a formulation to provide additional magnesium (Mg²⁺), an ion that is often needed for human nutrition but not abundantly found in food.

The quantity of chloride in compositions of the present disclosure may include, for example, about 1% to about 5%, about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, or about 80% to about 85% of the total dry weight of the components in the composition.

Compositions of the present disclosure may contain a chloride concentration of, for example, at least about 0.5 g/L, at least about 0.75 g/L, at least about 1 g/L, at least about 1.5 g/L, at least about 2 g/L, at least about 2.25 g/L, at least about 2.5 g/L, at least about 2.75 g/L, at least about 3 g/L, at least about 3.25 g/L, at least about 3.5 g/L, at least about 3.75 g/L, at least about 4 g/L, at least about 4.25 g/L, at least about 4.5 g/L, at least about 4.75 g/L, at least about 5 g/L, at least about 5.25 g/L, at least about 5.5 g/L, at least about 5.75 g/L, at least about 6 g/L, at least about 6.25 g/L, at least about 6.5 g/L, at least about 6.75 g/L, at least about 7 g/L, at least about 7.25 g/L, at least about 7.5 g/L, at least about 7.75 g/L, at least about 8 g/L, at least about 8.25 g/L, at least about 8.5 g/L, at least about 8.75 g/L, at least about 9 g/L, at least about 9.25 g/L, at least about 9.5 g/L, at least about 9.75 g/L, at least about 10 g/L, at least about 10.25 g/L, at least about 10.5 g/L, at least about 10.75 g/L, at least about 11 g/L, at least about 11.25 g/L, at least about 11.5 g/L, at least about 11.75 g/L, at least about 12 g/L, at least about 12.5 g/L, at least about 13 g/L, at least about 13.5 g/L, at least about 14 g/L, at least about 14.5 g/L, or at least about 15 g/L.

Compositions of the present disclosure may contain a chloride concentration of, for example, about 0.5 g/L to about 2.5 g/L, about 1 g/L to about 3 g/L, about 1.5 g/L to about 3.5 g/L, about 2 g/L to about 4 g/L, about 2.5 g/L to about 4.5 g/L, about 3 g/L to about 5 g/L, about 3.5 g/L to about 5.5 g/L, about 4 g/L to about 6 g/L, about 4.5 g/L to about 6.5 g/L, about 5 g/L to about 7 g/L, about 5.5 g/L to about 7.5 g/L, about 6 g/L to about 8 g/L, about 6.5 g/L to about 8.5 g/L, about 7 g/L to about 9 g/L, about 7.5 g/L to about 9.5 g/L, about 8 g/L to about 10 g/L, about 10.5 g/L to about 12.5 g/L, about 11 g/L to about 13 g/L, or about 13 g/L to about 15 g/L.

Compositions of the present disclosure may contain a chloride concentration of, for example, about 0.5 g/L to about 1 g/L, about 0.5 g/L to about 2 g/L, about 0.5 g/L to about 3 g/L, about 0.5 g/L to about 4 g/L, about 0.5 g/L to about 5 g/L, about 0.5 g/L to about 6 g/L, about 0.5 g/L to about 7 g/L, about 0.5 g/L to about 8 g/L, about 0.5 g/L to about 9 g/L, about 0.5 g/L to about 10 g/L, about 1 g/L to about 2 g/L, about 1 g/L to about 3 g/L, about 1 g/L to about 4 g/L, about 1 g/L to about 5 g/L, about 1 g/L to about 6 g/L, about 1 g/L to about 7 g/L, about 1 g/L to about 8 g/L, about 1 g/L to about 9 g/L, about 1 g/L to about 10 g/L, about 1.5 g/L to about 2 g/L, about 1.5 g/L to about 3 g/L, about 1.5 g/L to about 4 g/L, about 1.5 g/L to about 5 g/L, about 1.5 g/L to about 6 g/L, about 1.5 g/L to about 7 g/L, about 1.5 g/L to about 8 g/L, about 1.5 g/L to about 9 g/L, about 1.5 g/L to about 10 g/L, about 2 g/L to about 3 g/L, about 2 g/L to about 4 g/L, about 2 g/L to about 5 g/L, about 2 g/L to about 6 g/L, about 2 g/L to about 7 g/L, about 2 g/L to about 8 g/L, about 2 g/L to about 9 g/L, about 2 g/L to about 10 g/L, about 2.5 g/L to about 4 g/L, about 2.5 g/L to about 5 g/L, about 2.5 g/L to about 6 g/L, about 2.5 g/L to about 7 g/L, about 2.5 g/L to about 8 g/L, about 2.5 g/L to about 9 g/L, or about 2.5 g/L to about 10 g/L,

Compositions of the present disclosure may contain a chloride concentration of, for example, about 3 g/L to about 4 g/L, about 3 g/L to about 5 g/L, about 3 g/L to about 6 g/L, about 3 g/L to about 7 g/L, about 3 g/L to about 8 g/L, about 3 g/L to about 9 g/L, about 3 g/L to about 10 g/L, about 3.5 g/L to about 4 g/L, about 3.5 g/L to about 5 g/L, about 3.5 g/L to about 6 g/L, about 3.5 g/L to about 7 g/L, about 3.5 g/L to about 8 g/L, about 3.5 g/L to about 9 g/L, about 3.5 g/L to about 10 g/L, about 4 g/L to about 5 g/L, about 4 g/L to about 6 g/L, about 4 g/L to about 7 g/L, about 4 g/L to about 8 g/L, about 4 g/L to about 9 g/L, about 4 g/L to about 10 g/L, about 4.5 g/L to about 5 g/L, about 4.5 g/L to about 6 g/L, about 4.5 g/L to about 7 g/L, about 4.5 g/L to about 8 g/L, about 4.5 g/L to about 9 g/L, about 4.5 g/L to about 10 g/L, about 5 g/L to about 6 g/L, about 5 g/L to about 7 g/L, about 5 g/L to about 8 g/L, about 5 g/L to about 9 g/L, about 5 g/L to about 10 g/L, about 5.5 g/L to about 6 g/L, about 5.5 g/L to about 7 g/L, about 5.5 g/L to about 8 g/L, about 5.5 g/L to about 9 g/L, about 5.5 g/L to about 10 g/L, about 6 g/L to about 7 g/L, about 6 g/L to about 8 g/L, about 6 g/L to about 9 g/L, or about 6 g/L to about 10 g/L.

Compositions of the present disclosure may contain a chloride concentration of, for example, about 6.5 g/L to about 7 g/L, about 6.5 g/L to about 8 g/L, about 6.5 g/L to about 9 g/L, about 6.5 g/L to about 10 g/L, about 6.5 g/L to about 11 g/L, about 6.5 g/L to about 12 g/L, about 6.5 g/L to about 13 g/L, about 6.5 g/L to about 14 g/L, about 6.5 g/L to about 15 g/L, about 7 g/L to about 8 g/L, about 7 g/L to about 9 g/L, about 7 g/L to about 10 g/L, about 7 g/L to about 11 g/L, about 7 g/L to about 12 g/L, about 7 g/L to about 13 g/L, about 7 g/L to about 14 g/L, about 7 g/L to about 15 g/L, about 7.5 g/L to about 8 g/L, about 7.5 g/L to about 9 g/L, about 7.5 g/L to about 10 g/L, about 7.5 g/L to about 11 g/L, about 7.5 g/L to about 12 g/L, about 7.5 g/L to about 13 g/L, about 7.5 g/L to about 14 g/L, about 7.5 g/L to about 15 g/L, about 8 g/L to about 9 g/L, about 8 g/L to about 10 g/L, about 8 g/L to about 11 g/L about 8 g/L to about 12 g/L, about 8 g/L to about 13 g/L, about 8 g/L to about 14 g/L about 8 g/L to about 15 g/L, about 8.5 g/L to about 9 g/L, about 8.5 g/L to about 10 g/L, about 8.5 g/L to about 11 g/L, about 8.5 g/L to about 12 g/L, about 8.5 g/L to about 13 g/L, about 8.5 g/L to about 14 g/L, about 8.5 g/L to about 15 g/L, about 9 g/L to about 10 g/L, about 9 g/L to about 11 g/L, about 9 g/L to about 12 g/L, about 9 g/L to about 13 g/L, about 9 g/L to about 14 g/L, about 9 g/L to about 15 g/L, about 9.5 g/L to about 10 g/L, about 9.5 g/L to about 11 g/L, about 9.5 g/L to about 12 g/L, about 9.5 g/L to about 13 g/L, about 9.5 g/L to about 14 g/L, or about 9.5 g/L to about 15 g/L.

As described further herein, compositions of the present disclosure also contain potassium. Compositions of the present disclosure may contain a chloride concentration of, for example, about 25% to about 35%, about 30% to about 35%, about 35% to about 40%, about 40% to about 45%, about 45% to about 50%, about 50% to about 55%, about 55% to about 60%, about 60% to about 65%, or about 65% to about 70% of the total dry weight of the potassium and the chloride in the composition.

Organic Acid

In one aspect, consumable electrolyte compositions as described herein contain an organic acid. Organic acids are organic compounds (contain carbon) that have acidic properties. Organic acids include, for example, organic carboxylic acids. The presence of organic acids impacts the pH of compositions of the present disclosure. Various organic acids or their salts may be used in compositions of the present disclosure such as, for example, potassium citrate and citric acid. Various organic acids or their salts are commercially available and suitable organic acids or their salts for use in the compositions and methods described herein would be readily apparent to one of skill in the art in view of the present disclosure. Additional examples of potential organic acids include acetic acid in pure form or in the form of vinegar. Other short chain fatty acids, e.g. butyric acid, are also available.

Compositions of the present disclosure may contain an organic acid concentration of, for example, about 0.25 g/L to about 0.5 g/L, about 0.5 g/L to about 0.75 g/L, about 0.75 g/L to about 1 g/L, about 1 g/L to about 1.25 g/L, about 1.25 g/L to about 1.5 g/L, about 1.5 g/L to about 1.75 g/L, about 1.75 g/L to about 2 g/L, about 2 g/L to about 2.25 g/L, about 2.25 g/L to about 2.5 g/L, about 2.5 g/L to about 2.75 g/L, about 2.75 g/L to about 3 g/L, about 3 g/L to about 3.25 g/L, about 3.25 g/L to about 3.5 g/L, about 3.5 g/L to about 3.75 g/L, about 3.75 g/L to about 4 g/L, about 4 g/L to about 4.25 g/L, about 4.25 g/L to about 4.5 g/L, about 4.5 g/L to about 4.75 g/L, about 4.75 g/L to about 5 g/L, about 5 g/L to about 5.25 g/L, about 5.25 g/L to about 5.5 g/L, about 5.5 g/L to about 5.75 g/L, or about 5.75 g/L to about 6 g/L.

Compositions of the present disclosure may contain an organic acid concentration of, for example, at least about 0.25 g/L to about 1 g/L, about 0.25 g/L to about 2 g/L, about 0.25 g/L to about 3 g/L, about 0.25 g/L to about 4 g/L, about 0.25 g/L to about 5 g/L, about 0.5 g/L to about 1 g/L, about 0.5 g/L to about 2 g/L, about 0.5 g/L to about 3 g/L, about 0.5 g/L to about 4 g/L, about 0.5 g/L to about 5 g/L, about 1 g/L to about 1.5 g/L, about 1 g/L to about 1.75 g/L, about 1 g/L to about 2 g/L, about 1 g/L to about 3 g/L, about 1 g/L to about 4 g/L, about 1 g/L to about 5 g/L, about 1 g/L to about 6 g/L, about 1.5 g/L to about 2 g/L, about 1.5 g/L to about 2.5 g/L, about 1.5 g/L to about 3 g/L, about 1.5 g/L to about 4 g/L, about 1.5 g/L to about 5 g/L, about 1.5 g/L to about 6 g/L, about 2 g/L to about 3 g/L, about 2 g/L to about 3.5 g/L, about 2 g/L to about 4 g/L, about 2 g/L to about 5 g/L, about 2 g/L to about 6 g/L, about 2.5 g/L to about 3 g/L, about 2.5 g/L to about 3.5 g/L, about 2.5 g/L to about 4 g/L, about 2.5 g/L to about 5 g/L, about 2.5 g/L to about 6 g/L, about 3 g/L to about 4 g/L, about 3 g/L to about 4.5 g/L, about 3 g/L to about 5 g/L, about 3 g/L to about 6 g/L, about 3.5 g/L to about 4.5 g/L, about 3.5 g/L to about 5 g/L, about 3.5 g/L to about 6 g/L, about 4 g/L to about 5 g/L, about 4 g/L to about 6 g/L, or about 5 g/L to about 6 g/L.

Compositions of the present disclosure may contain an organic acid in an amount sufficient to achieve a particular pH. Compositions of the present disclosure may contain an organic acid in an amount sufficient to generate a pH of at least about 2.0, at least about 2.25, at least about 2.75, at least about 3.0, about least about 3.25, at least about 3.5, at least about 3.75, at least about 4.0, at least about 4.25, at least about 4.75, at least about 5.0, about least about 5.25, at least about 5.5, at least about 5.75, at least about 6.0, at least about 6.25, at least about 6.5, or at least about 6.75.

Compositions of the present disclosure may contain an organic acid in an amount sufficient to generate a pH of at least about 2.5, at least about 2.6, at least about 2.7, at least about 2.8, at least about 2.9, at least about 3.0, at least about 3.1, at least about 3.2, at least about 3.3, at least about 3.4, at least about 3.5, about least about 3.6, at least about 3.7, at least about 3.8, at least about 3.9, at least about 4.0, at least about 4.1, at least about 4.2, at least about 4.3, at least about 4.4, at least about 4.5, at least about 4.6, at least about 4.7, at least about 4.8, about least about 4.9, at least about 5.0, at least about 5.1, at least about 5.2, at least about 5.3, or at least about 5.4.

Compositions of the present disclosure may contain an organic acid in an amount sufficient to generate a pH of about 2.0 to about 2.5, about 2.0 to about 3.0, about 2.0 to about 3.5, about 2.0 to about 4.0, about 2.0 to about 4.5, about 2.0 to about 5.0, about 2.0 to about 5.5, about 2.0 to about 6.0, about 2.0 to about 6.5, about 2.5 to about 3.0, about 2.5 to about 3.5, about 2.5 to about 4.0, about 2.5 to about 4.5, about 2.5 to about 5.0, about 2.5 to about 5.5, about 2.5 to about 6.0, about 2.5 to about 6.5, about 3.0 to about 3.5, about 3.0 to about 4.0, about 3.0 to about 4.5, about 3.0 to about 5.0, about 3.0 to about 5.5, about 3.0 to about 6.0, about 3.0 to about 6.5, about 3.5 to about 4.0, about 3.5 to about 4.5, about 3.5 to about 5.0, about 3.5 to about 5.5, about 3.5 to about 6.0, about 3.5 to about 6.5, about 4.0 to about 4.5, about 4.0 to about 5.0, about 4.0 to about 5.5, about 4.0 to about 6.0, about 4.0 to about 6.5, about 4.5 to about 5.0, about 4.5 to about 5.5, about 4.5 to about 6.0, about 4.5 to about 6.5, about 5.0 to about 5.5, or about 5.0 to about 6.0.

The use of an organic acid to produce a composition of the present disclosure having a relatively low pH (e.g. 3.0 to 3.5) is effective at masking the metallic taste of compositions containing potassium chloride (KCl).

Additional Electrolytes

Compositions of the present disclosure may contain a variety of additional electrolytes. Such electrolytes may include, for example, sodium (Na⁺), calcium (Ca²⁺), magnesium (Mg²⁺), hydrogen phosphate (HPO₄²⁻), and hydrogen carbonate (HCO₃⁻). Compositions as described herein may contain one or more additional electrolytes in various combinations. Various electrolytes or their salts are commercially available and may be used in the compositions and methods described herein, as would be readily apparent to one of skill in the art in view of the present disclosure.

Compositions of the present disclosure may contain one or more of e.g. sodium (Na⁺), calcium (Ca²⁺), magnesium (Mg²⁺), hydrogen phosphate (HPO₄²⁻), and hydrogen carbonate (HCO₃⁻) at a concentration of, for example, about 0.25 g/L to about 0.5 g/L, about 0.5 g/L to about 0.75 g/L, about 0.75 g/L to about 1 g/L, about 1 g/L to about 1.25 g/L, about 1.25 g/L to about 1.5 g/L, about 1.5 g/L to about 1.75 g/L, about 1.75 g/L to about 2 g/L, about 2 g/L to about 2.25 g/L, about 2.25 g/L to about 2.5 g/L, about 2.5 g/L to about 2.75 g/L, about 2.75 g/L to about 3 g/L, about 3 g/L to about 3.25 g/L, about 3.25 g/L to about 3.5 g/L, about 3.5 g/L to about 3.75 g/L, about 3.75 g/L to about 4 g/L, about 4 g/L to about 4.25 g/L, about 4.25 g/L to about 4.5 g/L, about 4.5 g/L to about 4.75 g/L, about 4.75 g/L to about 5 g/L, about 5 g/L to about 5.25 g/L, about 5.25 g/L to about 5.5 g/L, about 5.5 g/L to about 5.75 g/L, or about 5.75 g/L to about 6 g/L.

Compositions of the present disclosure may contain one or more of e.g. sodium (Nat), calcium (Ca²⁺), magnesium (Mg²⁺), hydrogen phosphate (HPO₄²⁻), and hydrogen carbonate (HCO₃⁻) at a concentration of, for example, at least about 0.25 g/L to about 1 g/L, about 0.25 g/L to about 2 g/L, about 0.25 g/L to about 3 g/L, about 0.25 g/L to about 4 g/L, about 0.25 g/L to about 5 g/L, about 0.5 g/L to about 1 g/L, about 0.5 g/L to about 2 g/L, about 0.5 g/L to about 3 g/L, about 0.5 g/L to about 4 g/L, about 0.5 g/L to about 5 g/L, about 1 g/L to about 1.5 g/L, about 1 g/L to about 1.75 g/L, about 1 g/L to about 2 g/L, about 1 g/L to about 3 g/L, about 1 g/L to about 4 g/L, about 1 g/L to about 5 g/L, about 1 g/L to about 6 g/L, about 1.5 g/L to about 2 g/L, about 1.5 g/L to about 2.5 g/L, about 1.5 g/L to about 3 g/L, about 1.5 g/L to about 4 g/L, about 1.5 g/L to about 5 g/L, about 1.5 g/L to about 6 g/L, about 2 g/L to about 3 g/L, about 2 g/L to about 3.5 g/L, about 2 g/L to about 4 g/L, about 2 g/L to about 5 g/L, about 2 g/L to about 6 g/L, about 2.5 g/L to about 3 g/L, about 2.5 g/L to about 3.5 g/L, about 2.5 g/L to about 4 g/L, about 2.5 g/L to about 5 g/L, about 2.5 g/L to about 6 g/L, about 3 g/L to about 4 g/L, about 3 g/L to about 4.5 g/L, about 3 g/L to about 5 g/L, about 3 g/L to about 6 g/L, about 3.5 g/L to about 4.5 g/L, about 3.5 g/L to about 5 g/L, about 3.5 g/L to about 6 g/L, about 4 g/L to about 5 g/L, about 4 g/L to about 6 g/L, or about 5 g/L to about 6 g/L.

Compositions of the present disclosure may contain magnesium (Mg²) at a concentration of, for example, about 0.05 g/L to about 0.15 g/L, about 0.15 g/L to about 0.25 g/L, about 0.25 g/L to about 0.35 g/L, about 0.35 g/L to about 0.45 g/L, about 0.45 g/L to about 0.55 g/L, about 0.55 g/L to about 0.65 g/L, about 0.65 g/L to about 0.75 g/L, about 0.75 g/L to about 0.85 g/L, about 0.85 g/L to about 0.95 g/L, about 0.95 g/L to about 1.05 g/L, about 1.05 g/L to about 1.15 g/L, about 1.15 g/L to about 1.25 g/L, or about 1.25 g/L to about 1.35 g/L. Compositions of the present disclosure may contain magnesium (Mg²) at a concentration of, for example, at least about 0.05 g/L, at least about 0.1 g/L, at least about 0.15 g/L, at least about 0.20 g/L, at least about 0.25 g/L, at least about 0.3 g/L, at least about 0.35 g/L, at least about 0.4 g/L, at least about 0.45 g/L, at least about 0.5 g/L, at least about 0.55 g/L, or at least about 0.6 g/L or more.

Compositions of the present disclosure may contain magnesium (Mg²) from the source of, for example, magnesium chloride, magnesium lactate, magnesium sulfate, magnesium carbonate, magnesium glycinate, magnesium malate, magnesium taurate, magnesium orotate, magnesium citrate, magnesium oxide, or magnesium amino acid chelate. The source of magnesium may be anhydrous (non-hydrated) or hydrous (hydrated). For example, magnesium chloride may refer to the chemical compound with the formula MgCl₂, or its various hydrates with the formula MgCl₂(H₂O)ₓ The hydrated magnesium chloride may have different number of water ligands depending on the value of “x” in the formula MgCl₂(H₂O)ₓ For example, when x=6, the magnesium chloride is magnesium chloride hexahydrate.

Sweeteners

Compositions of the present disclosure may contain a sweetener compound. In some embodiments, the sweetener is a natural sweetener such as e.g. sucrose. In some embodiments, the sweetener is an artificial sweetener such as e.g. Advantame®. Mixtures of sweeteners, either natural or artificial, may also be used. Suitable sweeteners and combinations of sweeteners may be selected for the desired nutritional characteristics, taste profile, mouthfeel, and other organoleptic factors. Sweeteners may include high intensity sweeteners and/or natural high intensity sweeteners, including, for example, stevia extracts, steviol glycosides, steviosides, rebaudioside A, rebaudioside B, rebaudioside C, rebaudioside D, rebaudioside F, dulcoside A, rubusosides, steviolbiosides, sucrose, high fructose corn syrup, fructose, fructooligosaccharides, glucose, xylose, arabinose, rhamnose, erythritol, xylitol, mannitol, sorbitol, inositol, AceK, aspartame, neotame, oligofructose, sucralose, saccharine, naringin dihydrochalcone (NarDHC), neohesperidin dihydrochalcone (NDHC), rubusoside, mogroside IV, siamenoside I, mogroside V, monatin, thaumatin, monellin, brazzein, L-alanine, glycine, hernandulcin, phyllodulcin, trilobtain, and combinations thereof. Various sweeteners are known in the art, are commercially available, and may be used in the compositions and methods described herein, as would be readily apparent to one of skill in the art in view of the present disclosure.

In embodiments where a sweetener is added to compositions described herein, the quantity of sweetener added is preferably minimal e.g. below 15% RDI (recommended daily intake).

The quantity of a sweetener compound in the compositions of the present disclosure may be, for example, equivalent to about 1% RDI, about 2% RDI, about 3% RDI, about 4% RDI, about 5% RDI, about 6% RDI, about 7% RDI, about 8% RDI, about 9% RDI, about 10% RDI, about 11% RDI, about 12% RDI, about 13% RDI, about 14% RDI, or about 15% RDI. Most preferably, the quantity of a sweetener compound is at or below about 6% RDI.

Compositions of the present disclosure may contain a sweetener compound at a concentration of, for example, about 1 g/L to about 5 g/L, about 5 g/L to about 7.5 g/L, about 7.5 g/L to about 10 g/L, about 10 g/L to about 12.5 g/L, about 12.5 g/L to about 15 g/L, about 15 g/L to about 17.5 g/L, about 17.5 g/L to about 20 g/L, about 20 g/L to about 22.5 g/L, about 22.5 g/L to about 25 g/L, about 25 g/L to about 27.5 g/L, about 27.5 g/L to about 30 g/L, about 30 g/L to about 32.5 g/L, about 32.5 g/L to about 35 g/L, about 35 g/L to about 37.5 g/L, about 37.5 g/L to about 40 g/L, about 40 g/L to about 45 g/L, about 45 g/L to about 50 g/L, about 50 g/L to about 55 g/L, about 55 g/L to about 60 g/L, about 60 g/L to about 65 g/L, about 65 g/L to about 70 g/L, or about 70 g/L to about 75 g/L.

Compositions of the present disclosure may contain a sweetener compound at a concentration of, for example, about 1 g/L to about 10 g/L, about 10 g/L to about 20 g/L, about 20 g/L to about 30 g/L, about 30 g/L to about 40 g/L, about 40 g/L to about 50 g/L, about 50 g/L to about 60 g/L, about 60 g/L to about 70 g/L, about 15 g/L to about 25 g/L, about 15 g/L to about 30 g/L, about 15 g/L to about 40 g/L, about 15 g/L to about 60 g/L, about 20 g/L to about 30 g/L, about 20 g/L to about 40 g/L, about 20 g/L to about 50 g/L, about 20 g/L to about 60 g/L, about 30 g/L to about 45 g/L, about 30 g/L to about 50 g/L, about 30 g/L to about 65 g/L, about 45 g/L to about 55 g/L, about 45 g/L to about 65 g/L, or about 50 g/L to about 70 g/L.

Orally Consumable Products

The consumable electrolyte compositions of the present disclosure may be formulated into an orally consumable product. As used herein, the term “orally consumable product(s)” refers to edible substances which are contacted with the mouth of an animal e.g. a human, including substances which are drunk, eaten, swallowed, or otherwise ingested; and that are safe for human or other animal consumption when used in a generally acceptable range of concentrations. The orally consumable products formulated from consumable electrolyte compositions may be administered to a subject to alleviate muscle cramping in the subject.

Orally consumable products of the present disclosure may take various forms. An orally consumable product of the present disclosure may include, for example, a foodstuff composition, a beverage product, a dietary supplement, a nutraceutical, an edible gel mix, and an edible gel composition. Orally consumable products may also be formulated into a granulated powder, a soft gel composition, and a flash dissolve composition.

Orally consumable products of the present disclosure may contain one or more additives. The one or more additives may be present to add or enhance one or more characteristics of the orally consumable product, such as flavor, texture, aroma, color, shelf life, etc. The one or more additives may already be present in the orally consumable product or may be added to the orally consumable product, or one or more compounds or ingredients used to make the orally consumable product. The orally consumable product may contain various suitable additives known in the art. Examples of suitable additives include, for example, carbohydrates, polyols, amino acids or salts thereof, poly-amino acids or salt thereof, sugar acids or salts thereof, nucleotides, organic acids, inorganic acids, organic salts, organic acid salts, organic base salts, inorganic salts, bitter compounds, flavorants, flavoring ingredients, astringent compounds, proteins, protein hydrolysates, surfactants, emulsifiers, flavonoids, alcohols, polymers, preservatives, thickening agents, food colorings, and combinations thereof.

Beverage Products

Consumable electrolyte compositions of the present disclosure may be formulated as a beverage product. Beverage products of the present disclosure include both carbonated and non-carbonated beverage products. Examples of suitable beverage products include, for example, soft drinks, fountain beverages, frozen beverages, ready-to-drink beverages, coffee, teas, dairy beverages, powdered soft drinks, liquid concentrates, flavored water, enhanced water, fruit juices, fruit juice flavored drinks, sport drinks, and energy drinks.

In some embodiments, a beverage product of the present disclosure includes one or more beverage ingredients including, for example, acidulants, fruit juices and/or vegetable juices, pulp, etc., flavorings, coloring, preservatives, vitamins, minerals, erythritol, tagatose, glycerine, and carbon dioxide. Such beverage products may be provided in any suitable form, such as a beverage concentrate or a carbonated or non-carbonated, ready-to-drink beverage.

Beverage products of the present disclosure may have numerous different specific formulations or constitutions. The formulation of a beverage product of the present disclosure may vary to a certain extent, depending upon such factors as the product's intended market segment, its desired nutritional characteristics, flavor profile, and the like. For example, in certain embodiments, it will generally be an option to add further ingredients to the formulation of a particular beverage product. For example, sweeteners, flavorings, vitamins, fruit juices or other fruit products, tastents, masking agents and the like, flavor enhancers, and/or carbonation typically may be added to any such formulations to vary the taste, mouthfeel, nutritional characteristics, etc. In some embodiments, beverage products are formulated to exhibit a particular flavor(s). Flavors that may be used may include, for example, vanilla flavor, chocolate flavor, banana flavor, strawberry flavor, lemon lime flavor, fruit punch flavor, orange flavor, raspberry flavor, and various others that will be readily apparent to one of skill in the art. Exemplary additional flavorings include, for example, cola flavoring, citrus flavoring, and spice flavorings. In some embodiments, carbonation in the form of carbon dioxide may be added for effervescence. In other embodiments, preservatives may be added, depending upon the other ingredients, production technique, desired shelf life, etc. In certain embodiments, caffeine may be added. In some embodiments, the beverage product is a cola-flavored carbonated beverage, characteristically containing carbonated water, sweetener, kola nut extract and/or other flavoring, caramel coloring, one or more acids, and optionally other ingredients. In some embodiments, the beverage product is a flavored, non-carbonated sports drink.

Dietary Supplements and Nutraceuticals

Consumable electrolyte compositions of the present disclosure may be formulated into a dietary supplement product. As used herein, “dietary supplement(s)” refers to compounds intended to supplement the diet and provide nutrients, such as vitamins, minerals, fiber, fatty acids, amino acids, etc. that may be missing or may not be consumed in sufficient quantities in a diet or may not be consumed in sufficient quantities during a particular event or activity (e.g. an athletic activity). Various suitable dietary supplements are known in the art and may be used in the compositions described herein. Examples of suitable dietary supplements include, for example, nutrients, vitamins, minerals, fiber, fatty acids, herbs, botanicals, amino acids, and metabolites.

Consumable electrolyte compositions of the present disclosure may be formulated into a nutraceutical product. As used herein, “nutraceutical(s)” refers to compounds, which includes any food or part of a food that may provide medicinal or health benefits, including the prevention and/or treatment of disease or disorder (e.g., fatigue, effects of aging, muscle cramping, etc.). Various suitable nutraceuticals are known in the art and may be used in the compositions described herein. In some embodiments, nutraceuticals can be used as supplements to food and beverages and as pharmaceutical formulations for enteral or parenteral applications which may be solid formulations, such as capsules or tablets, or liquid formulations, such as solutions or suspensions.

In some embodiments, dietary supplements and nutraceuticals may further contain protective hydrocolloids (such as gums, proteins, modified starches), binders, film-forming agents, encapsulating agents/materials, wall/shell materials, matrix compounds, coatings, emulsifiers, surface active agents, solubilizing agents (oils, fats, waxes, lecithins, etc.), adsorbents, carriers, fillers, co-compounds, dispersing agents, wetting agents, processing aids (solvents), flowing agents, taste-masking agents, weighting agents, jellyfying agents, gel-forming agents, antioxidants, and antimicrobials.

Edible Gel Mixes and Gel Compositions

Consumable electrolyte compositions of the present disclosure may be formulated as gel mixes and gel compositions. As used herein, a “gel” refers to a colloidal system in which a network of particles spans the volume of a liquid medium. Although gels mainly are composed of liquids, and thus exhibit densities similar to liquids, gels have the structural coherence of solids due to the network of particles that spans the liquid medium. For this reason, gels generally appear to be solid, jelly-like materials. Gels that can be eaten are referred to as “edible gel compositions.” Edible gel compositions typically are eaten as snacks, as performance-enhancing supplements, as desserts, as a part of staple foods, or along with staple foods. Examples of suitable edible gel compositions include, for example, gel desserts, puddings, jams, jellies, pastes, trifles, aspics, marshmallows, gummy candies, and the like. In some embodiments, edible gel mixes generally are powdered or granular solids to which a fluid may be added to form an edible gel composition. Examples of suitable fluids include, for example, water, dairy fluids, dairy analogue fluids, juices, and combinations thereof. Examples of suitable dairy fluids include, for example, milk, cultured milk, cream, fluid whey, and mixtures thereof. Examples of suitable dairy analogue fluids include, for example, soy milk and non-dairy coffee whitener.

As used herein, the term “gelling ingredient” refers to any material that can form a colloidal system within a liquid medium. Examples of suitable gelling ingredients include, for example, gelatin, alginate, carageenan, gum, pectin, konjac, agar, food acid, rennet, starch, starch derivatives, and combinations thereof. It is well known to those having ordinary skill in the art that the amount of gelling ingredient used in an edible gel mix or an edible gel composition varies considerably depending on a number of factors including, for example, the particular gelling ingredient used, the particular fluid base used, and the desired properties of the gel.

Gel mixes and gel compositions of the present disclosure may be prepared by various suitable methods known in the art. In some embodiments, edible gel mixes and edible gel compositions may contain various ingredients. Examples of other suitable ingredients include, for example, a food acid, a salt of a food acid, a buffering system, a bulking agent, a sequestrant, a cross-linking agent, one or more flavors, one or more colors, and combinations thereof.

### Alleviating Muscle Cramping

The present disclosure provides methods of alleviating muscle cramping in a subject. Methods of alleviating muscle cramping in a subject generally involve administering to the subject a consumable electrolyte composition of the present disclosure.

In some embodiments, the subject is administered a consumable electrolyte composition containing potassium in the range of about 2 g/L to about 8 g/L, chloride in the range of about 2 g/L to about 8 g/L, an organic acid in an amount sufficient to generate a pH in the range of about 3.0 to about 3.5, and where the potassium is present in the range of about 45% to about 55% of the total dry weight of the potassium and the chloride in the composition.

### Muscle Cramping

Muscle cramps arise from sudden and involuntary contractions in various muscle tissues. These cramps are almost universally uncomfortable and/or painful to some degree, but severe muscle cramps can be quite painful and even debilitating. There are various causes of muscle cramps, including electrolyte imbalance or deficiency in the muscle that is the source of the cramping.

The consumable electrolyte compositions of the present disclosure may be administered to a subject to alleviate muscle cramping. Alleviation of muscle cramping may refer to, for example, reducing the number instances of muscle cramping in an individual over a time period, wholly or partially reducing the onset of muscle cramping (preventative), reducing the severity/intensity of muscle cramping, and reducing the duration of a muscle cramp event. Exemplary muscle cramps to be alleviated may include, for example, leg muscle cramps, including hamstring cramps, foot cramps, and menstrual cramps.

Subjects

Various subjects may be administered a consumable electrolyte composition of the present disclosure to alleviate muscle cramping. Suitable subjects would be readily understood by one of skill in the art in view of the present disclosure. Suitable subjects include, for example, any animal with muscle tissue, such as a human.

The subject may be a male or female human individual that engages in athletic activity. Athletic activity, depending on the level of intensity, typically results in loss of electrolytes from the body which may lead to the development of muscle cramping. Individuals engaging in athletic activity (e.g. athletes) are thus suitable subjects for administering consumable electrolyte compositions of the present disclosure.

The subject may be a male or female human individual that is elderly (e.g. over 65+ years in age). Body function, including muscle function, tends to decrease with increasing age, which may lead to the development of muscle cramping in elderly individuals. Elderly individuals are thus suitable subjects for administering consumable electrolyte compositions of the present disclosure. Elderly individuals may be e.g. at least 50 years old, at least 55 years old, at least 60 years old, at least 65 years old, at least 70 years old, at least 75 years old, at least 80 years old, or at least 85 years old. One of skill in the art would readily recognize elderly individuals.

Administering Consumable Electrolyte Compositions

Certain methods of the present disclosure may involve administering an effective amount of a consumable electrolyte composition of the present disclosure to a subject. The term “effective amount” as used herein refers to an amount of a compound or composition sufficient to alleviate, ameliorate, palliate, mitigate, lessen, delay, and/or eliminate one or more symptoms of a disorder, condition, or disease such as e.g. muscle cramping. An effective amount of a consumable electrolyte composition may or may not be achieved in conjunction with another drug, pharmaceutical composition, or treatment. Thus, an effective amount may be considered in the context of administering one or more therapeutic agents, and a single agent may be considered to be given in an effective amount if, in conjunction with one or more other agents, a desirable result may be or is achieved.

To alleviate muscle cramping in a subject, the appropriate dosage of a consumable electrolyte composition of the present disclosure (when used alone or in combination with one or more other additional therapeutic agents) may depend on, for example, the type of muscle cramp to be treated or prevented, the severity and course of the muscle cramp if already present in the subject, whether the consumable electrolyte composition is administered for preventative or therapeutic purposes, previous therapy and type of therapy received by the subject, the subject's clinical history, and the discretion of any attending physician or sports trainer, if applicable.

The dosage of a consumable electrolyte composition of the present disclosure may be administered to a subject at one time or over a series of administrations over a time interval. For example, the consumable electrolyte composition may be administered to a subject once daily, twice daily, three or more times daily, once every two days, once every three days, once every four days, once every five days, once every six days, once every week, once every two weeks, or once every month or longer. For repeated administrations over several days or longer, depending on the specific conditions of the subject, the administration would generally be sustained until a desired suppression of muscle cramp symptoms occurs or until there is sufficient confidence that muscle cramps will not appear or that any muscle cramps will have reduced severity upon appearance. The duration of the administration of the consumable electrolyte composition may be, for example, at least one day, at least three days, at least one week, at least two weeks, at least one month, at least two months, at least three months, at least six months, at least nine months, or at least one year or longer.

The progress of administration regimens of consumable electrolyte compositions are easily monitored by conventional techniques and assays. In some embodiments, the progress of the administration is monitored by monitoring changes in onset, frequency, severity, duration, etc. of muscle cramping events.

The quantity of a consumable electrolyte composition of the present disclosure that is administered during each instance of administration may include, for example, at least about 0.05 L, 0.1 L, at least about 0.2 L, at least about 0.3 L, at least about 0.4 L, at least about 0.5 L, at least about 0.6 L, at least about 0.7 L, at least about 0.8 L, at least about 0.9 L, or at least about 1 L of the consumable electrolyte composition.

The quantity of a consumable electrolyte composition of the present disclosure that is administered during each instance of administration may include, for example, about 0.05 L to about 0.1 L, about 0.1 L to about 0.2 L, about 0.2 L to about 0.3 L, about 0.3 L to about 0.4 L, about 0.4 L to about 0.5 L, about 0.5 L to about 0.6 L, about 0.6 L to about 0.7 L, about 0.7 L to about 0.8 L, about 0.8 L to about 0.9 L, or about 0.9 L to about 1 L of the consumable electrolyte composition.

The quantity of a consumable electrolyte composition of the present disclosure that is administered during each instance of administration may include, for example, about 0.05 L to about 0.2 L, about 0.05 L to about 0.4 L, about 0.05 L to about 0.6 L, about 0.05 L to about 0.8 L, about 0.1 L to about 0.3 L, about 0.1 L to about 0.5 L, about 0.1 L to about 0.7 L, about 0.1 L to about 1 L, about 0.3 L to about 0.5 L, about 0.3 L to about 0.6 L, about 0.3 L to about 0.7 L, about 0.7 L to about 1 L, about 0.4 L to about 0.6 L, about 0.4 L to about 0.8 L, about 0.4 L to about 0.9 L, about 0.4 L to about 1 L, about 0.5 L to about 0.7 L, about 0.5 L to about 0.8 L, about 0.5 L to about 0.9 L, about 0.5 L to about 1 L, about 0.6 L to about 0.8 L, about 0.6 L to about 1 L, about 0.7 L to about 1 L, or about 0.8 L to about 1 L of the consumable electrolyte composition.

Consumable electrolyte compositions of the present disclosure may manifest various degrees of alleviation of muscle cramping in subjects who have consumed these compositions. For example, reducing the number instances of muscle cramping in an individual over a time period, wholly or partially reducing the onset of muscle cramping (preventative), reducing the severity/intensity of muscle cramping, and/or reducing the duration of a muscle cramp event may refer to, for example, a reduction of muscle cramping symptoms of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 95% reduction of muscle cramping symptoms following consumption of a consumable electrolyte composition of the present disclosure.

Consumable electrolyte compositions of the present disclosure may alleviate muscle cramping in a subject within a certain period of time following initial consumption of the consumable electrolyte composition. Alleviation of muscle cramping in a subject may occur, for example, about 30 seconds, about 45 seconds, about 1 minute, about 1.5 minutes, about 2 minutes, about 2.5 minutes, about 3 minutes, about 3.5 minutes, about 4 minutes, about 4.5 minutes, about 5 minutes, about 6 minutes, about 7 minutes, about 8 minutes, about 9 minutes, about 10 minutes, about 12.5 minutes, about 15 minutes, about 20 minutes, about 30 minutes, about 45 minutes, about 1 hour, about 1.5 hours, or about 2 or more hours after consumption of the consumable electrolyte composition.

Alleviation of muscle cramping in a subject may occur, for example, about 30 seconds to about 45 seconds, about 30 seconds to about 1 minute, about 30 seconds to about 2 minutes, about 30 seconds to about 3 minutes, about 30 seconds to about 4 minutes, about 30 seconds to about 5 minutes, about 30 seconds to about 7.5 minutes, about 30 seconds to about 10 minutes, about 30 seconds to about 30 minutes, about 30 seconds to about 1 hour, about 1 minute to about 5 minutes, about 1 minute to about 10 minutes, about 1 minute to about 30 minutes, about 1 minute to about 45 minutes, about 1 minute to about 1 hour, about 5 minutes to about 10 minutes, about 5 minutes to about 30 minutes, about 5 minutes to about 1 hour, about 10 minutes to about 30 minutes, about 10 minutes to about 45 minutes, about 10 minutes to about 1 hour, about 10 minutes to about 2 hours, about 15 minutes to about 30 minutes, about 15 minutes to about 1 hour, or about 15 minutes to about 2 hours after consumption of the consumable electrolyte composition.

In some embodiments, the reduction of muscle cramping symptoms in individuals who have been administered a consumable electrolyte composition of the present disclosure may be compared to muscle cramping symptoms in a control subject. One of skill in the art would readily recognize various control subjects that would be suitable for such comparisons. For example, the control subject may be a comparable individual to the subject individual that does not experience muscle cramping. The control subject may be an individual that is administered a different consumable electrolyte composition (e.g. having a different potassium concentration) than the subject under evaluation. One of skill in the art would readily recognize an appropriate control subject in view of the present disclosure.

Kits

Also provided herein are kits for alleviating muscle cramping in a subject. Kits of the present disclosure contain a consumable electrolyte composition as described herein.

In some embodiments, the kit contains instructions for administering a consumable electrolyte composition of the present disclosure to a subject. In some embodiments, the consumable electrolyte composition is formulated into an orally consumable product of the present disclosure. The instructions may provide information related to, for example, suggested guidelines for dosing regimens and duration of administration of the consumable electrolyte composition. The consumable electrolyte composition present in the kit may also be provided in a package. Consumable electrolyte compositions of the present disclosure may be provided in, for example, a paper package, a plastic package, a bottle, or one of various other suitable packages well known to those of skill in the art.

Menstrual Cramping

A menstrual cramp is a specific type of muscle cramp caused by contraction of uterine muscle. Menstrual cramps, also known as dysmenorrhea or painful periods, manifest as throbbing pains in the lower abdomen during menstruation. Symptoms usually begin around the time that menstruation begins, and can last for two to four days. The pain associated with menstrual cramping can range from dull and annoying to severe and extreme. Menstrual cramping may be caused by identifiable medical problems, such as endometriosis or uterine fibroids. Without wishing to be bound by theory, it is thought that an unbalanced electrolyte profile in the body may also contribute to menstrual cramping. The consumable electrolyte compositions of the present disclosure may be administered to a subject to alleviate menstrual cramping as described above.

Various subjects may be administered a consumable electrolyte composition of the present disclosure to alleviate menstrual cramping. Suitable subjects would be readily understood by one of skill in the art in view of the present disclosure. Suitable subjects include, for example, any animal that experiences menstruation, such as a human. The subject is typically a female human individual. Female individuals may be of various ages, ranging from adolescents to the elderly. Female individuals may be e.g. at least 10 years old, at least 15 years old, at least 20 years old, at least 25 years old, at least 30 years old, at least 35 years old, at least 40 years old, at least 45 years old, at least 50 years old, at least 55 years old, or at least 60 years old.

### EXAMPLES

The following examples are offered for illustrative purposes and to aid one of skill in better understanding the various embodiments of the disclosure. The following examples are not intended to limit the scope of the present disclosure in any way.

### Example 1: Consumable Electrolyte Composition Formulations

This Example demonstrates exemplary formulations of various consumable electrolyte formulations. These formulations may be used in alleviating muscle cramping in a subject.

### Introduction

The majority of electrolyte-replacement beverages available on the market have formulations for electrolyte replacement and hydration that are based on the composition of sweat. However, it seems that this is based on the assumption that all of the electrolytes lost in sweat provide a complete picture of electrolyte homeostasis. However, without wishing to be bound by theory, it is thought that this assumption does not provide the complete picture of electrolyte homeostasis.

Without wishing to be bound by theory, Applicant believes that the sweat-centric view does not take into account the physiology of the shift in kidney function during sweating. As the body loses sodium through sweat, the kidney shifts from losing sodium to losing potassium, resulting in a loss of potassium from muscles as they function. The more an individual sweats, the more potassium is lost and how much a person sweats is generally related to the level of muscle function. This is important because, under normal conditions, potassium goes outside of the muscle when it contracts and then is pumped back into the muscle. However, as the kidney shifts to potassium wasting while sweating, it puts potassium into the urine, thus taking it out of the blood. This results in less potassium going back into the muscle cell. When the potassium stores in the muscle cell are sufficiently depleted, cramping occurs.

Accordingly, Applicant has developed a series of consumable electrolyte compositions that may act to deliver potassium to the muscles to alleviate muscle cramping. These compositions are formulated to provide an exogenous source of potassium into the body to shift the potassium homeostasis and enhance its entry into muscle cells. The compositions also contain chloride which is beneficial in driving water entry into cells and promoting hydration. The formulations of these compositions are described below.

Formulations

Formulations 1-3 in Tables 1-3 below, respectively, illustrate electrolyte beverages containing ˜1 gram of potassium per 500 mL serving (0.5 L, brought to volume with water). Formulations 1-3 are formulated to have a lemon-lime flavor, an orange flavor, or a fruit punch flavor, respectively.

TABLE 1
Formulation 1

| | | Calories | |
|---|---|---|---|
| | | or Grams | % RDI |
| Ingredients per 500 mL | Amount | (g) | (recommended |
| serving | (grams) | electrolyte | daily intake) |
| KCl | 1.655 g | K = 0.87 g | 50% |
| Kcitrate (potassium citrate) | 0.35 g | K = 0.127 g | 3% |
| Sucrose | 25 g | 96.7 | 12% |
| NaCl | 0.51 g | Na = 0.20 g | 7% |
| Citric acid | 1.05 g | | |
| Natural lemon-lime flavor | 0.145 g | | |
| (Weber) | | | |

TABLE 2
Formulation 2

| | | Calories | |
|---|---|---|---|
| | | or Grams | % RDI |
| Ingredients per 500 mL | Amount | (g) | (recommended |
| serving | (grams) | electrolyte | daily intake) |
| KCl | 1.655 g | K = 0.87 g | 25% |
| Kcitrate (potassium citrate) | 0.35 g | K = 0.127 g | 3% |
| Sucrose | 25 g | 96.75 | 12% |
| NaCl | 0.51 g | Na = 0.20 g | 7% |
| Citric acid | 0.86 g | | |
| Natural Orange Flavor | 0.125 g | | |
| (Weber) | | | |

TABLE 3
Formulation 3

| | | Calories | |
|---|---|---|---|
| | | or Grams | % RDI |
| Ingredients per 500 mL | Amount | (g) | (recommended |
| serving | (grams) | electrolyte | daily intake) |
| KCl | 1.655 g | K = 0.87 g | 25% |
| Kcitrate (potassium citrate) | 0.35 g | K = 0.127 g | 3% |
| Sucrose | 25 g | 96.75 | 12% |
| NaCl | 0.51 g | Na = 0.20 g | 7% |
| Citric acid | 0.69 g | | |
| Natural Fruit Punch Flavor | 0.125 g | | |
| (Weber) | | | |

Formulations 4-6 in Tables 4-6 below, respectively, are similar to Formulations 1-3, except that Formulations 4-6 contain a reduced amount of sucrose (lower calorie beverage).

TABLE 4
Formulation 4

| | | Calories | |
|---|---|---|---|
| | | or Grams | % RDI |
| Ingredients per 500 mL | Amount | (g) | (recommended |
| serving | (grams) | electrolyte | daily intake) |
| KCl | 1.56 g | K = 0.82 g | 25% |
| Kcitrate (potassium citrate) | 0.48 g | K = 0.18 g | 3% |
| 0.01% Advantame ® in | 10 g | 48.375 | 6% |
| Sucrose | | | |
| NaCl | 0.51 g | Na = 0.20 g | 7% |
| Citric acid | 1.05 g | | |
| Natural lemon-lime flavor | 0.125 g | | |
| (Weber) | | | |

TABLE 5
Formulation 5

| | | Calories | |
|---|---|---|---|
| | | or Grams | % RDI |
| Ingredients per 500 mL | Amount | (g) | (recommended |
| serving | (grams) | electrolyte | daily intake) |
| KCl | 1.56 g | K = 0.82 g | 25% |
| Kcitrate (potassium citrate) | 0.48 g | K = 0.18 g | 3% |
| 0.01% Advantame ® in | 12.5 g | 48.375 | 6% |
| Sucrose | | | |
| NaCl | 0.51 g | Na = 0.20 g | 7% |
| Citric acid | 0.86 g | | |
| Natural Orange Flavor | 0.125 g | | |
| (Weber) | | | |

TABLE 6
Formulation 6

| | | Calories | |
|---|---|---|---|
| | | or Grams | % RDI |
| Ingredients per 500 mL | Amount | (g) | (recommended |
| serving | (grams) | electrolyte | daily intake) |
| KCl | 1.56 g | K = 0.82 g | 25% |
| Kcitrate (potassium citrate) | 0.48 g | K = 0.18 g | 3% |
| 0.01% Advantame ® in | 12.5 g | 48.375 | 6% |
| Sucrose | | | |
| NaCl | 0.51 g | Na = 0.20 g | 7% |
| Citric acid | 0.69 g | | |
| Natural Fruit Punch Flavor | 0.125 g | | |
| (Weber) | | | |

Formulations 7-9 in Tables 7-9 below, respectively, illustrate electrolyte beverages containing ˜2 grams of potassium per 500 mL serving (0.5 L, brought to volume with water). Formulations 7-9 are formulated to have a lemon-lime flavor, an orange flavor, or a fruit punch flavor, respectively.

TABLE 7
Formulation 7

| | | Calories | |
|---|---|---|---|
| | | or Grams | % RDI |
| Ingredients per 500 mL | Amount | (g) | (recommended |
| serving | (grams) | electrolyte | daily intake) |
| KCl | 3.56 g | K = 1.87 g | 50% |
| Kcitrate (potassium citrate) | 0.35 g | K = 0.13 g | 3% |
| Sucrose | 30 g | 116.1 | 14% |
| NaCl | 0.51 g | Na = 0.20 g | 7% |
| Citric acid | 1.05 g | | |
| Natural lemon-lime flavor | 0.145 g | | |
| (Weber) | | | |

TABLE 8
Formulation 8

| | | Calories | |
|---|---|---|---|
| | | or Grams | % RDI |
| Ingredients per 500 mL | Amount | (g) | (recommended |
| serving | (grams) | electrolyte | daily intake) |
| KCl | 3.56 g | K = 1.87 g | 50% |
| Kcitrate (potassium citrate) | 0.35 g | K = 0.13 g | 3% |
| Sucrose | 30 g | 116.1 | 14% |
| NaCl | 0.51 g | Na = 0.20 g | 7% |
| Citric acid | 0.86 g | | |
| Natural Orange Flavor | 0.125 g | | |
| (Weber) | | | |

TABLE 9
Formulation 9

| | | Calories | |
|---|---|---|---|
| | | or Grams | % RDI |
| Ingredients per 500 mL | Amount | (g) | (recommended |
| serving | (grams) | electrolyte | daily intake) |
| KCl | 3.56 g | K = 1.87 g | 50% |
| Kcitrate (potassium citrate) | 0.35 g | K = 0.13 g | 3% |
| Sucrose | 30 g | 116.1 | 14% |
| NaCl | 0.51 g | Na = 0.20 g | 7% |
| Citric acid | 0.685 g | | |
| Natural Fruit Punch Flavor | 0.125 g | | |
| (Weber) | | | |

Formulations 10-12 in Tables 10-12 below, respectively, are similar to Formulations 7-9, except that Formulations 10-12 contain a reduced amount of sucrose (lower calorie beverage).

TABLE 10
Formulation 10

| | | Calories | |
|---|---|---|---|
| | | or Grams | % RDI |
| Ingredients per 500 mL | Amount | (g) | (recommended |
| serving | (grams) | electrolyte | daily intake) |
| KCl | 3.465 g | K = 1.82 g | 50% |
| Kcitrate (potassium citrate) | 0.48 g | K = 0.18 g | 3% |
| 0.01% Advantame ® in | 15 g | 58.05 | 7% |
| Sucrose | | | |
| NaCl | 0.51 g | Na = 0.20 g | 7% |
| Citric acid | 1.05 g | | |
| Natural lemon-lime flavor | 0.145 g | | |
| (Weber) | | | |

TABLE 11
Formulation 11

| | | Calories | |
|---|---|---|---|
| | | or Grams | % RDI |
| Ingredients per 500 mL | Amount | (g) | (recommended |
| serving | (grams) | electrolyte | daily intake) |
| KCl | 3.465 g | K = 1.82 g | 50% |
| Kcitrate (potassium citrate) | 0.48 g | K 0.18 g | 3% |
| 0.01% Advantame ® in | 15 g | 58.05 | 7% |
| Sucrose | | | |
| NaCl | 0.51 g | Na = 0.20 g | 7% |
| Citric acid | 0.86 g | | |
| Natural Orange Flavor | 0.125 g | | |
| (Weber) | | | |

TABLE 12
Formulation 12

| | | Calories | |
|---|---|---|---|
| | | or Grams | % RDI |
| Ingredients per 500 mL | Amount | (g) | (recommended |
| serving | (grams) | electrolyte | daily intake) |
| KCl | 3.465 g | K = 1.82 g | 50% |
| Kcitrate (potassium citrate) | 0.48 g | K = 0.18 g | 3% |
| 0.01% Advantame ® in | 15 g | 58.05 | 7% |
| Sucrose | | | |
| NaCl | 0.51 g | Na = 0.20 g | 7% |
| Citric acid | 0.69 g | | |
| Natural Fruit Punch Flavor | 0.125 g | | |
| (Weber) | | | |

Formulation 13 below illustrates an electrolyte beverage containing ˜4 grams of potassium per 500 mL serving (0.5 L, brought to volume with water). Formulation 13 is formulated to have a lemon-lime flavor.

TABLE 13
Formulation 13

| | | Calories | |
|---|---|---|---|
| | | or Grams | % RDI |
| Ingredients per 500 mL | Amount | (g) | (recommended |
| serving | (grams) | electrolyte | daily intake) |
| KCl | 7.284 g | K = 3.82 g | 99% |
| Kcitrate (potassium citrate) | 0.48 g | K = 0.18 g | 4% |
| Sucrose | 30 g | 116.1 | 14% |
| NaCl | 0.51 g | Na = 0.20 g | 7% |
| Citric acid | 0.48 g | | |
| Natural lemon-lime flavor | 0.25 g | | |
| (Weber) | | | |

Formulation 14 in Table 14 below is similar to Formulation 13, except that Formulation 14 contains a reduced amount of sucrose (lower calorie beverage).

TABLE 14
Formulation 14

| | | Calories | |
|---|---|---|---|
| | | or Grams | % RDI |
| Ingredients per 500 mL | Amount | (g) | (recommended |
| serving | (grams) | electrolyte | daily intake) |
| KCl | 7.284 g | K = 3.82 g | 99% |
| Kcitrate (potassium citrate) | 0.48 g | K = 0.18 g | 4% |
| 0.01% Advantame ® in | 20 g | 77.4 | 9% |
| Sucrose | | | |
| NaCl | 0.51 g | Na = 0.20 g | 7% |
| Citric acid | 0.96 g | | |
| Natural lemon-lime flavor | 0.25 g | | |
| (Weber) | | | |

The consumable electrolyte formulations described in this Example may find use in alleviating muscle cramping in a subject. These formulations contain a rapidly absorbable form of potassium and may be used to quickly and efficiently restore normal muscle function. In particular, these formulations may be used to rapidly mitigate acute muscle cramping, as well as to be used prophylactically to help prevent or reduce chronic muscle cramping as well. Without wishing to be bound by theory, the formulations containing ˜1 g-2 g of potassium per 500 mL serving may preferably be used to act as a prophylactic of muscle cramping and help prevent chronic recurrence, whereas the formulations containing greater than ˜2 g of potassium per 500 mL serving (e.g. containing ˜4+ g of potassium per 500 mL serving) may preferably be used to mitigate the acute onset of muscle cramps.

### Example 2: Use of Consumable Electrolyte Compositions to Alleviate Muscle Cramping

This Example describes an analysis of individual subjects who have consumed various consumable electrolyte formulations as described in Example 1. Each act of consumption of a particular formulation in this Example refers to consuming one 500 mL serving of the respective formulation described. The subjects consuming these formulations reported alleviation of their muscle cramping.

Subject 1: A 77-year-old male who consumes one of Formulations 7-9 (a Formulation having ˜2 g potassium per serving) twice daily to prevent onset of nighttime leg and foot cramps.

Subject 2: A 64-year-old male who consumes one of Formulations 7-9 (a Formulation having ˜2 g potassium per serving) once or twice daily to prevent onset of nighttime leg and foot cramps.

Subject 3: A 53-year-old male who consumes one of Formulations 13-14 (a Formulation having ˜4 g potassium per serving) four times a day to prevent chronic cramping and onset of acute cramping.

Subject 4: A 20-year-old male who consumes one of Formulations 13-14 (a Formulation having ˜4 g potassium per serving) 5-7 times a day to prevent chronic cramping and onset of acute cramping. This individual is a NCAA DI wrestler.

Subject 5: A 17-year-old male who consumes one of Formulations 7-12 (a Formulation having ˜2 g potassium per serving) twice daily to prevent cramps. This individual is a high school football player.

Subject 6: A 20-year-old male who consumes one of Formulations 7-12 (a Formulation having ˜2 g potassium per serving) to prevent cramps, and who consumes one of Formulations 13-14 (a Formulation having ˜4 g potassium per serving) to stop acute onset cramps during competition. This individual is a NCAA DI hockey goalie.

Subject 7: A 21-year-old male who consumes one of Formulations 7-12 (a Formulation having ˜2 g potassium per serving) and Formulations 13-14 (a formulation containing ˜4 g potassium per serving) to prevent cramps during competition and to maintain bodyweight during competition. This individual is a NCAA DI hockey defenseman.

Subjects 8-16: These subjects are 19-23 year-old males who consume one of Formulations 7-12 (a Formulation having ˜2 g potassium per serving) t and Formulations 13-14 (a formulation containing ˜4 g potassium per serving) to prevent cramps during competition. These individuals are NCAA DI hockey players.

Subject 17: A 26-year-old male who consumes one of Formulations 7-12 (a Formulation having ˜2 g potassium per serving) to prevent cramps during competition.

Subject 18: A 36-year-old male who consumes one of Formulations 7-12 (a Formulation having ˜2 g potassium per serving) to prevent cramps during competition.

This study highlights the ability of the formulations described in Example 1 (containing ˜1-4 g of potassium per 500 mL serving) to alleviate muscle cramping in a variety of subjects. In some subjects, alleviation of muscle cramping began to manifest in as little as 30 seconds following initial consumption of the electrolyte formulations 13-14 (a formulation containing ˜4 g potassium per serving).

### Example 3: Clinical Analysis of Consumable Electrolyte Compositions for Alleviation of Nighttime Muscle Cramping

This Example provides an outline of a study designed to confirm the effectiveness of various consumable electrolyte compositions to alleviate nighttime muscle cramping.

Introduction

The onset of nighttime (nocturnal) muscle cramps in elderly individuals is a known phenomenon. These nighttime muscle cramps tend to occur in the leg muscles, in particular the calf muscles or small foot muscles. These muscle cramps can be particularly unsettling in elderly individuals, and these cramps are actually quite common in the elderly population. In a study of 233 people aged 60+, approximately one third of those participating in the study reported experiencing a rest cramping event in the previous two months. For those individuals who were 80+ years old, 50% of respondents reported experiencing such cramps. Along the same lines, in another study of 350 elderly outpatients, it was found that 50% of those surveyed reported having rest cramps, and 20% of respondents reported having cramping symptoms that have lasted for 10+ years (Butler et al., 2002, “Nocturnal leg cramps in older people,” Postgrad Med J2002, 78:596-598).

As described in Example 2, several elderly individuals reported that various consumable electrolyte formulations as described in Example 1 were effective at alleviating their nighttime muscle cramps. This present study is designed to confirm the effectiveness of the formulations described in Example 1 at alleviating nighttime muscle cramps in elderly individuals.

Study Outline

Study population: Approximately 30 or more patients of age 60+.

Eligibility criteria: Subjects will be eligible for inclusion in the study if they meet the criteria associated with the “study population” and have reported that they have experienced regular or semi-regular incidents of nighttime muscle cramping for at least the past two months.

Exclusion criteria: Subjects deemed to have a clinical disorder or status that would be incompatible with the present study will be excluded from the study. Subjects deemed by a health care professional to be unfit for the study will be excluded from the study.

Baseline Characterization

Initial baseline characterization of the subjects will be performed to analyze various aspects of nighttime muscle cramping in the subject, including characterizing the severity of the cramping and the history of the cramping.

Study Groups

Subjects will be blindly divided into two groups: 1) those selected to receive one of formulations 1-14 as described in Example 1, and 2) those selected to receive a control formulation. Various control formulations may be considered. For example, the control formulation may be a commercially available electrolyte beverage that contains a significantly lower concentration of potassium than any of formulations 1-14 (e.g. Gatorade, which contains 45 mg of potassium in a 12 oz. serving). The control formulation may be a beverage that contains all of the components of one of formulations 1-14, but does not contain the potassium component, or contains a substantially reduced amount of potassium as compared to the potassium concentration of formulations 1-14. The control formulation may be a sweetened water beverage that does not contain any potassium, or contains a substantially reduced amount of potassium as compared to the potassium concentration of formulations 1-14.

Treatment Regimen

Study subjects will be instructed to consume one serving (500 mL) of their designated beverage (either one of formulations 1-14 or a control beverage) at night before they go to bed. This consumption at this time should be done every single day for the duration of the study period. The study period may last for one week, two weeks, three weeks, one month, two months, three months, or longer depending on various factors.

Treatment Analysis

At the end of the study period, subjects will be analyzed to characterize the effectiveness of the respective beverage designated to them at alleviating nighttime muscle cramps. Subjects will be asked to assess various factors, including the incidence of nighttime muscle cramps during the study period, the severity of such cramps (if applicable), the duration of such cramps (if applicable), etc.

Once the relevant data has been gathered, an analysis will be performed to determine the effectiveness of one of formulations 1-14 as compared to the control formulations at alleviating nighttime muscle cramps.

Efficacy Outcomes

It is expected that formulations 1-14 of Example 1 will provide greater alleviation of nighttime muscle cramping in elderly subjects as compared to control formulations.

### Example 4: Survey Analysis of Consumable Electrolyte Compositions for Alleviation of Cramping

This Example provides an analysis of a human subject survey to probe the effectiveness of consumable electrolyte compositions at alleviating a formed cramp and at reducing the severity of cramping in surveyed subjects after they became consumers of the consumable electrolyte compositions.

Introduction

In research of human subjects, a survey is a list of questions aimed at extracting specific data from a particular group of people. Survey research may be used to obtain data on the responses of a group of subjects towards a certain product. Data gathered from a subject survey therefore provides valuable information that could be used to evaluate the effectiveness of a product.

As previously described, individuals reported that various consumable electrolyte formulations as noted in Example 1 were effective at alleviating their muscle cramps. This study was designed to confirm the effectiveness of various formulations described in Example 1 at alleviating formed cramps, as well as at reducing the severity of cramps when they do occur in individuals. These outcomes were assessed based on subject responses to inquiries in terms of that response before they became consumers of the consumable electrolyte compositions, and that response after they became consumers of the consumable electrolyte compositions.

Methods

A total of 60 individuals were surveyed in an anonymous manner through a third-party survey service provider. Survey subjects used various consumable electrolyte formulations (depending on the particular subject), including: Formulations 1 and 4 (referred to as 1K), Formulations 7-10 (referred to as 2K), and Formulations 13 and 14 (referred to as 4K) as described in Example 1. Subjects were asked about the severity of their cramps before they became consumers of the consumable electrolyte compositions, as well as the severity of their cramps after they became consumers of the consumable electrolyte compositions. Severity of cramping was rated on a 0-10 scale, with 0 indicating no cramps and 10 indicating debilitating cramps. 34 surveys were completed, with 31 fully completed and three partially completed, yielding a total approximate response rate of 57%.

Results

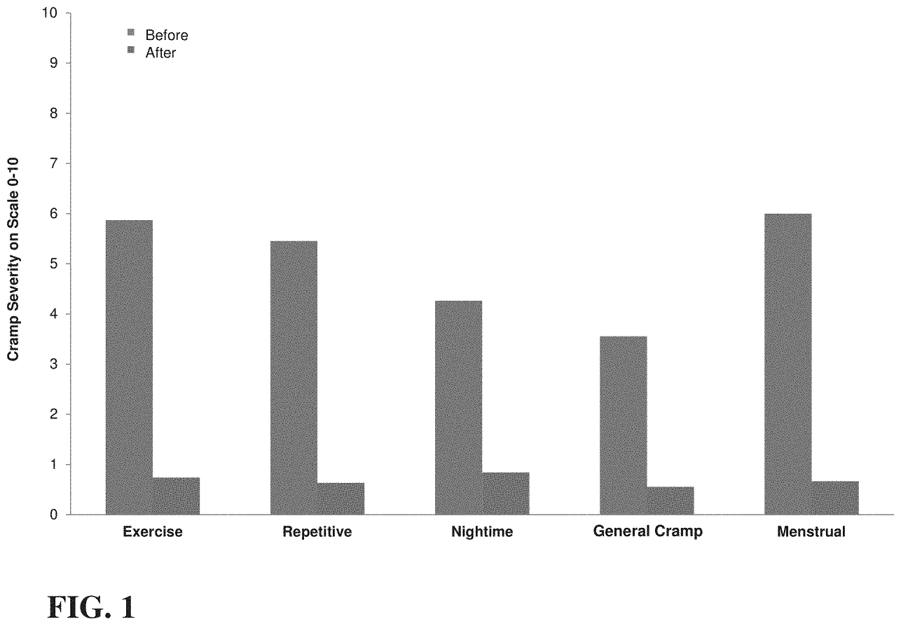

Survey results are described below, and data are shown in FIG. 1 to FIG. 7 and in Table 15 and Table 16.

One prong of the analysis of the survey results was to determine if the respondents experienced relief from their cramping after becoming consumers of the consumable electrolyte compositions. Table 15 below illustrates the effectiveness of consumable electrolyte compositions at providing relief from various types of cramps, including exercise-induced, repetitive use, nighttime leg and foot, general, and menstrual cramps. Results showed that 85.7-90% of respondents reported full effectiveness of the consumable electrolyte compositions, 10-14.3% of respondents reported partial effectiveness of the consumable electrolyte compositions, and 0% of respondents reported ineffectiveness of the consumable electrolyte compositions. Many respondents noted a complete mitigation of cramps within less than 5 minutes, while others had complete relief after becoming consumers of these compositions (e.g. they no longer had cramps at all)(See also FIG. 1-FIG. 6).

TABLE 15
Effectiveness of consumable electrolyte compositions at remediating various cramps

| Type of Cramp | Complete Relief | Partial Relief | No Relief | Respondents |
|---|---|---|---|---|
| Exercise Induced Cramps | 18 | 3 | 0 | 21 |
| Repetitive Use Cramps | 9 | 1 | 0 | 10 |
| Nighttime Leg and Foot Cramps | 14 | 2 | 0 | 16 |
| General Cramping | 7 | 1 | 0 | 8 |
| Menstrual Cramps | 2 | 0 | 0 | 2 |

| | % Complete | % Partial | % No Relief |
|---|---|---|---|
| Exercise Induced Cramps | 85.7% | 14.3% | 0.0% |
| Repetitive Use Cramps | 90.0% | 10.0% | 0.0% |
| Nighttime Leg and Foot Cramps | 87.5% | 12.5% | 0.0% |
| General Cramping | 87.5% | 12.5% | 0.0% |
| Menstrual Cramps | 100.0% | 0.0% | 0.0% |

Data on the severity of various cramps in individuals both before and after they became consumers of the electrolyte compositions was used to graphically illustrate the change in severity of these cramps after becoming a consumer of the electrolyte compositions. Table 16 below shows the raw data of respondents' cramp severities before and after they became consumers of the consumable electrolyte compositions, where 0=no cramping and 10=debilitating cramps.

TABLE 16
Survey respondents’ cramp severities before and after becoming consumers of
the consumable electrolyte compositions

| | Exercise Induced | | Repetitive Use | | Nighttime | | General | | Menstrual | |
|---|---|---|---|---|---|---|---|---|---|---|
| Cramp | Cramps | | Cramps | | Cramps | | Cramping | | Cramps | |
| Severity | Before | After | Before | After | Before | After | Before | After | Before | After |
| 0 | 0 | 12 | 0 | 6 | 0 | 12 | 0 | 6 | 0 | 1 |
| 1 | 2 | 7 | 1 | 3 | 7 | 2 | 5 | 2 | 0 | 0 |
| 2 | 0 | 3 | 1 | 2 | 2 | 2 | 0 | 0 | 0 | 1 |
| 3 | 2 | 0 | 1 | 0 | 1 | 2 | 0 | 1 | 0 | 0 |
| 4 | 5 | 1 | 1 | 0 | 0 | 0 | 1 | 0 | 0 | 0 |
| 5 | 1 | 0 | 1 | 0 | 0 | 0 | 0 | 0 | 0 | 0 |
| 6 | 2 | 0 | 1 | 0 | 1 | 0 | 0 | 0 | 0 | 0 |
| 7 | 5 | 0 | 3 | 0 | 0 | 0 | 2 | 0 | 1 | 0 |
| 8 | 2 | 0 | 1 | 0 | 6 | 0 | 0 | 0 | 1 | 0 |
| 9 | 1 | 0 | 0 | 0 | 1 | 0 | 1 | 0 | 0 | 0 |
| 10 | 3 | 0 | 1 | 0 | 0 | 0 | 0 | 0 | 0 | 0 |
| Total | 23 | 23 | 11 | 11 | 18 | 18 | 9 | 9 | 2 | 2 |

Respondents

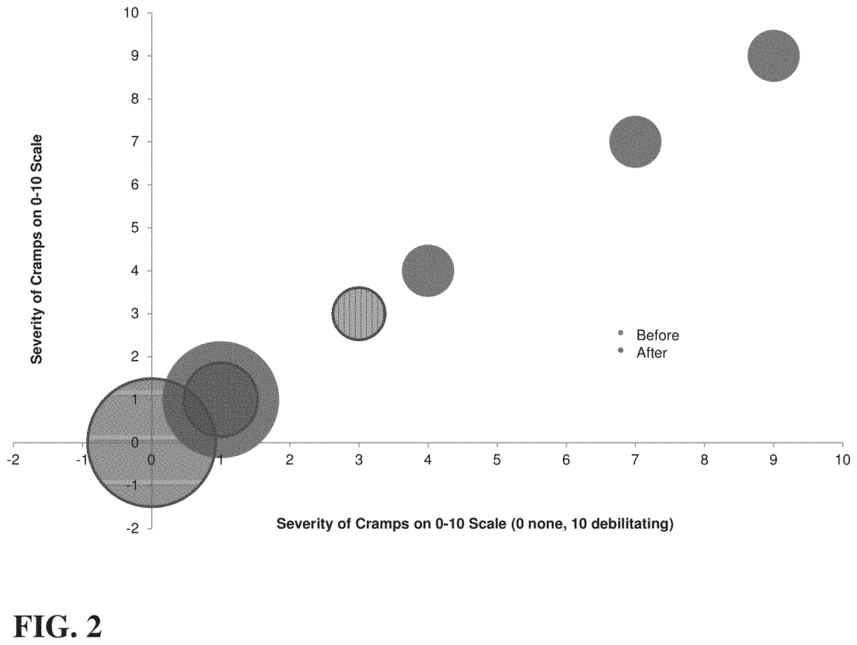

The data in Table 16 are presented in various graphical formats in FIG. 1 to FIG. 6 to graphically illustrate the change in severity of cramping in surveyed individuals after they became consumers of the electrolyte compositions (compared to severity of cramping before they were consumers of these compositions). In FIG. 1 to FIG. 6, blue indicates severity of cramps before respondents became consumers of the consumable electrolyte compositions, and red indicates severity of cramps after respondents became consumers of the consumable electrolyte compositions. The size of circles in FIG. 2 to FIG. 6 represents the number of individuals at a given level of severity for a given cramp type. Respondents varied from individuals who lightly cramp to individuals with debilitating cramps, and these scores reflect the average of a large dynamic range.

FIG. 1 shows the average severity ratings before and after subjects became consumers of the consumable electrolyte composition for the different types of cramps, including exercise-induced, repetitive use, nighttime, general and menstrual cramps. For all types of cramps, the consumable electrolyte composition provided a marked reduction in cramping severity from 4-6 to 0-1.

For general cramping (FIG. 2), respondents reported a severity rating range of 1-9 before they became consumers of the electrolyte compositions. After they became consumers of the consumable electrolyte compositions, respondents reported drastic decreases of cramping severity: ratings of cramping severity dropped to the range of 0-3, with the majority of individuals having a severity rating of 0, meaning no longer having cramps at all.

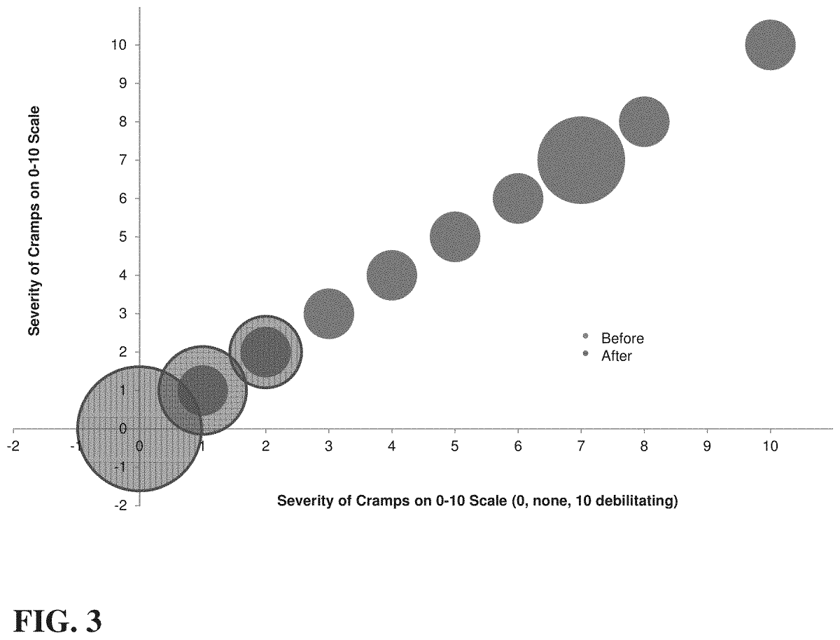

For repetitive use cramps (FIG. 3), respondents reported a severity rating range of 1-10 before they became consumers of the consumable electrolyte compositions. After they became consumers of the consumable electrolyte compositions, respondents reported drastic decreases of cramping severity: ratings of severity dropped to the range of 0-2. Similar to general cramping, the majority of individuals reported a severity rating of 0, indicating they stopped having cramps completely after becoming consumers of the consumable electrolyte compositions.

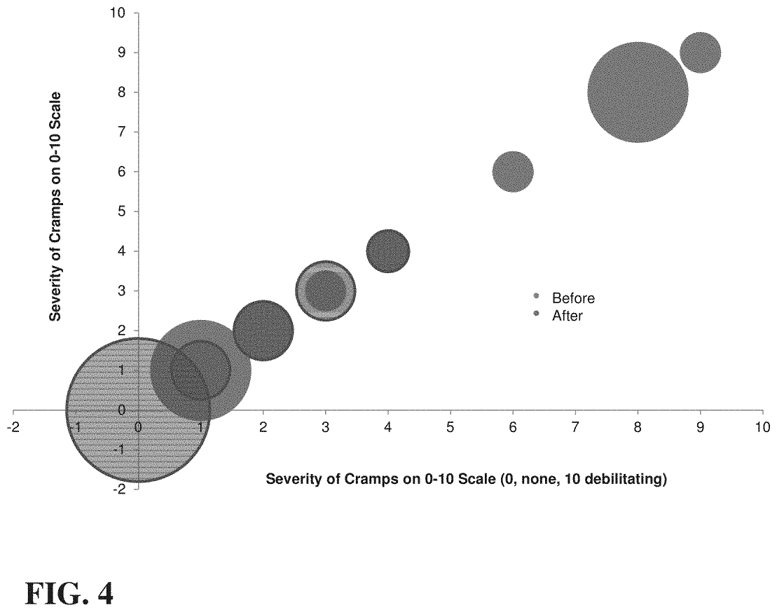

As shown in FIG. 4, subjects suffering from nighttime leg and foot cramps also experienced great relief of cramps after they became consumers of the consumable electrolyte compositions. Severity ratings of cramps dropped from the range of 1-9 to the range of 0-4, with a large number of respondents reporting a rating of 0 after becoming consumers of the consumable electrolyte compositions, indicating they stopped suffering from nighttime leg and foot cramps altogether.

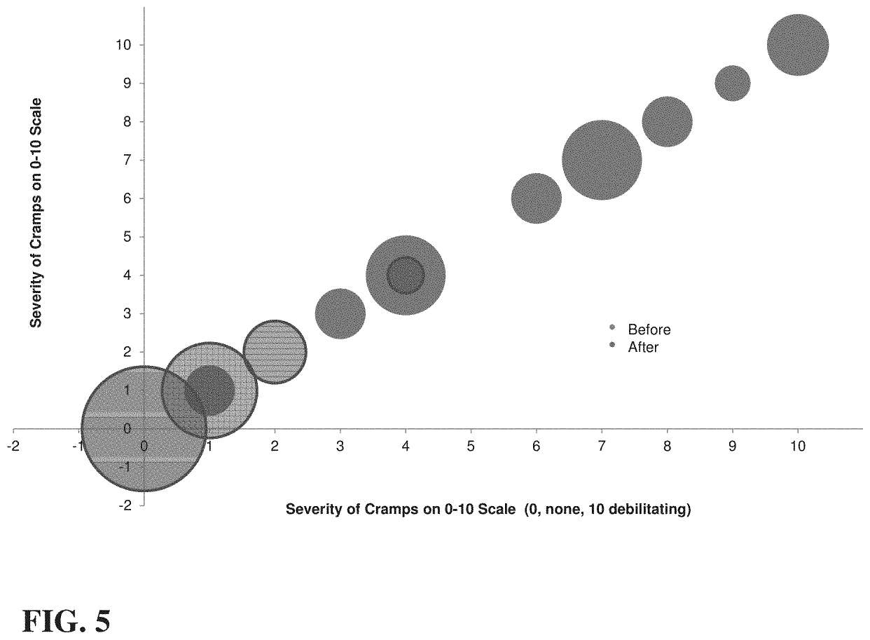

FIG. 5 shows that for exercise-induced cramps, before becoming consumers of the consumable electrolyte compositions, the respondents were spread between severity ratings of 1-10, with a large number of individuals rating their severities above 7. After becoming consumers of the consumable electrolyte compositions, cramps for the majority of subjects went to 0-4 in severity, demonstrating effective cramp prevention and, when cramps did occur, the severity and pain associated with the cramping was greatly reduced. Also noted is that a large number of individuals stopped suffering from cramps altogether with a severity rating of 0 after becoming consumers of the consumable electrolyte compositions.

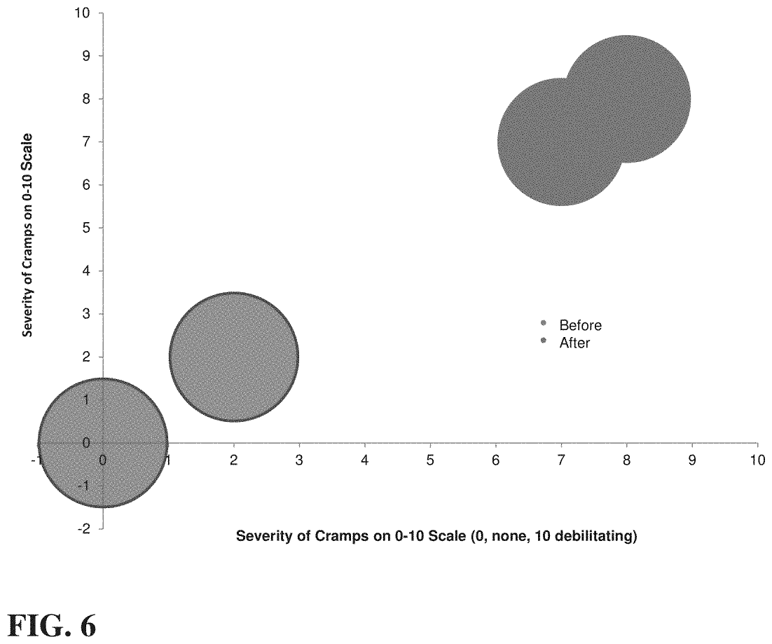

For individuals with menstrual cramps, two female subjects responded to this type of cramping. As indicated in FIG. 6, the severity of the menstrual cramps dropped significantly from 7-8 to 0-2 after these respondents became consumers of the consumable electrolyte compositions. Further, both female respondents reported that their menstrual cramps were mitigated within 5 minutes or less after using the consumable electrolyte compositions.

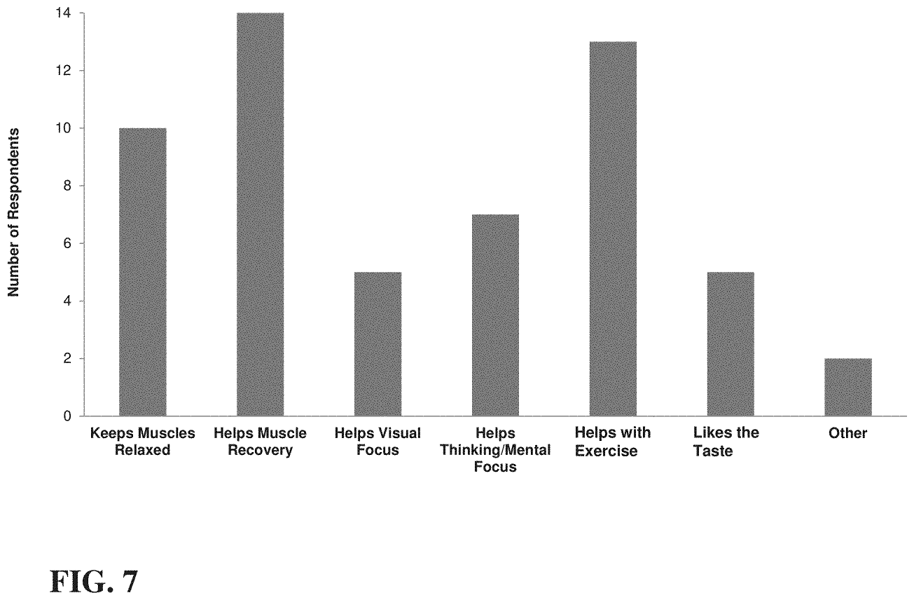

In addition to remediating cramps, surveyed individuals noted additional positive attributes of the consumable electrolyte compositions. The number of respondents for each category is noted in FIG. 7. The most significant positive attributes are that using consumable electrolyte composition keeps muscles relaxed (10), helps muscle recovery (14), and helps with exercise (13).

Conclusions

Overall, the survey described in this Example demonstrates that the consumable electrolyte compositions described herein reduce the severity of cramps and reduce the incidence of the occurrence of cramps in surveyed subjects. Further, most respondents reported that they experienced cramp remediation within 5 minutes or less of consuming the electrolyte composition, with many experiencing relief in one minute or less. The results demonstrate the effectiveness of using the consumable electrolyte compositions described herein on a regular basis to keep cramping either under control or alternatively to shift an individual to not having cramps at all.

Indeed, that 85.7-90% of respondents (8-21 respondents) experienced complete relief from cramping across 4 different categories of cramps is remarkable. Respondents reported that after becoming consumers of the electrolyte compositions, severity of cramps was reduced when cramps did occur, and many respondents stopped having cramps altogether.

1-19: (canceled)

20. A method of alleviating muscle cramping in a human subject, the method comprising:
a) providing a human subject experiencing a muscle cramp;
b) administering to the subject a liquid consumable electrolyte composition in an amount sufficient to alleviate the muscle cramp in 5 minutes or less, wherein the electrolyte composition comprises:
potassium in the range of about 4 g/L to about 8 g/L, wherein the source of potassium consists of potassium chloride and potassium citrate;
chloride in the range of about 3.5 g/L to about 7.5 g/L, wherein the source of chloride consists of potassium chloride and sodium chloride;
sodium in an amount up to about 0.5 g/L;
citric acid in the range of about 1 g/L to about 2.5 g/L, and;
a sweetener selected from the group consisting of sucrose, advantame, rebaudioside D, and stevia extracts, and;
wherein the quantity of potassium is present in the range of about 45% to about 55% of the total dry weight of the potassium and the chloride in the electrolyte composition;
wherein the quantity of potassium accounts for at least about 40% to about 55% of the total dry weight of the electrolytes in the electrolyte composition;
wherein the pH of the electrolyte composition is in the range of about 3.0 to about 3.5;
wherein the electrolyte composition does not exhibit a metallic flavor profile;
c) maintaining the subject under conditions such that the muscle cramp is alleviated in 5 minutes or less following administration of the electrolyte composition.

21. The method of claim 20, wherein the amount is in the range of about 0.3 L to about 0.6 L.

22. The method of claim 21, wherein the amount is about 0.5 L.

23. The method of claim 20, wherein the composition is administered over a time interval.

24. The method of claim 20, wherein the composition is administered about once daily.

25. The method of claim 20, wherein the muscle cramp is a leg cramp, a foot cramp, or a menstrual cramp.

26. The method of claim 20, wherein alleviation comprises reducing the duration of the muscle cramp and/or reducing the severity of the muscle cramp.

27. (canceled)

28. The method of claim 20, wherein the human subject is an athlete or an elderly individual.

29. The method of claim 20, wherein the human subject is a female.

30. The method of claim 20, wherein the potassium is in the range of about 4 g/L to about 6 g/L.

31. The method of claim 20, wherein the potassium is in the range of about 6 g/L to about 8 g/L.

32. The method of claim 20, wherein the chloride is in the range of about 4 g/L to about 6 g/L.

33. The method of claim 20, wherein the chloride is in the range of about 6 g/L to about 7.5 g/L.

34. The method of claim 20, wherein the sodium is in the range of about 0.25 g/L to about 0.5 g/L.

35. The method of claim 20, wherein the composition further comprises magnesium in the range of about 0.05 g/L to about 0.45 g/L.

36. The method of claim 35, wherein the source of magnesium is magnesium chloride.
